SEQUENCE LISTING (12 sequences)

SEQ ID NO: 1 - protein, 25 aa, Artificial Sequence
  Description of Artificial Sequence Xenopus laevis axin residues 380-404
DIHVDPEKFAAELISRLEGVLRDRE

SEQ ID NO: 2 - protein, 25 aa, Artificial Sequence
  Description of Artificial Sequence Chick axin residues 380-404
DIHVEPEKFAAELINRLEEVQKERE

SEQ ID NO: 3 - protein, 25 aa, Artificial Sequence
  Description of Artificial Sequence Murine axin residues 509-532
EIRVEPQKFAEELIHRLEAVQRTRE

SEQ ID NO: 4 - protein, 25 aa, Artificial Sequence
  Description of Artificial Sequence Human axin residues 417-440
EVRVEPQKFAEELIHRLEAVQRTRE

SEQ ID NO: 5 - protein, 25 aa, Artificial Sequence
  Description of Artificial Sequence Human axin 2 residues 362-386
MTPVEPATFAAELISRLEKLKLELE

SEQ ID NO: 6 - protein, 25 aa, Artificial Sequence
  Description of Artificial Sequence Rat axil residues 362-386
MTPVEPAAFAAELISRLEKLKLELE

SEQ ID NO: 7 - protein, 25 aa, Artificial Sequence
  Description of Artificial Sequence Murine conductin residues 362-386
MTPVEPAAFAAELISRLEKLKLELE

SEQ ID NO: 8 - protein, 842 aa, Xenopus laevis
MSVKGKGFPLDLGGSFTEDAPRPPVPGEEGELITTDQRPFSHTYYSLKNDGIKNETSTATPRRPDLDLGYEPEGSASPTPPYLKWAESLHSLLDDQDGIHLFRTFLQQENCADLLDFWFACSGFRKLEPNDSKVEKRLKLAKAIYKKYVLDSNGIVSRQIKPATKSFIKDCVLRQQIDPAMFDQAQMEIQSMMEDNTYPVFLKSDIYLEYTTIGGESPKNYSDQSSGSGTGKGPSGYLPTLNEDEEWRCDQGGEHERERECIPSSLFSQKLALDSSSHCAGSNRRLSDGREFRPGTWREPVNPYYVNTGYAGAPVTSANDSEQQSMSSDADTMSLTDSSVDGIPPYRLRKHYRREMQESANANGRGPLPHIPRTYHMPKDIHVDPEKFAAELISRLEGVLRDREAEQKLEERLKRVRAEEEGDDGDVSSGPSVISHKLPSGPPMHHFNSRYSETGCVGMQIRDAHEENPESILDEHVQRVMKTPGCQSPGTGRHSPKSRSPDGHLSKTLPGSLGTMQTGHGKHSSKSTAKVDSGNLHHHKHVYHHVHHHGGVKPKEQIDGESTQRVQTNFPWNVESHNYATKSRNYAESMGMAPNPMDSLAYSGKVSMLSKRNAKKADLGKSESASHEMPVVPEDSERHQKILQWIMEGEKEIIRHKKSNHSSSSAKKQPPTELARPLSIERPGAVHPWVSAQLRNVVQPSHPFIQDPTMPPNPAPNPLTQLVSKPGARLEEEEKKAAKMPQKQRLKPQKKNVSAPSQPCDNIVVAYYFCGEPIPYRTMVKGRVVTLGQFKELLTKKGNYRYYFKKVSDEFDCGVVFEEVREDDMILPIYEEKIIGQVEKID

SEQ ID NO: 9 - protein, 22 aa, Artificial Sequence
  Description of Artificial SequenceGID Consensus Sequence
VXPXXFAXELIXRLEXXXXXXE

SEQ ID NO: 10 - protein, 25 aa, Artificial Sequence
  Description of Artificial SequenceGID Consensus Sequence
XXXVXPXXFAXELIXRLEXXXXXXE

SEQ ID NO: 11 - protein, 25 aa, Artificial Sequence
  Description of Artificial SequenceGID Consensus Sequence
XXXVXPXXFAXELIXRLEXXXXXXE

SEQ ID NO: 12 - protein, 25 aa, Artificial Sequence
  Description of Artificial SequenceGID Consensus Sequence
XXXVXPXXFAXELIXRLEXXXXXXE

(19) United States
(12) Patent Application Publication
(10) Pub. No.: US 2001/0052137 A1
(43) Pub. Date: Dec. 13, 2001

(54) Axin domain-like polypeptide inhibitors of glycogen synthase kinase 3 beta activity and activators of wnt signaling

(75) Inventor: Peter S. Klein, Wynnewood, PA (US)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: Trustees of the University of Pennsylvania

(21) Appl. No.: 09/798,831

(22) Filed: Mar. 1, 2001

Related U.S. Application Data
(60) Provisional application No. 60/186,141, filed on Mar. 1, 2000.

(51) Int. Cl. A01K 67/027; A61K 38/55; C12N 9/99

(52) U.S. Cl. 800/18; 514/12; 435/184

(57) ABSTRACT

The invention relates to polypeptides which inhibit the activity of glycogen synthase kinase-3 beta (GSK-3 beta) in vivo and which also activate wnt signaling. The polypeptides have an amino acid sequence which includes one or both of an axin/GSK-3 beta interaction domain (GID) and an axin/axin interaction domain (AID). These polypeptides are useful for treating a number of disorders (e.g. bipolar disorder, mania, depression, Alzheimer's disease, diabetes, and leukopenia) which are presently treated by administration of lithium. The invention also includes antibodies (including fragments of antibodies) which bind specifically with the polypeptides described in the disclosure, and to transgenic mice which comprise a transgene encoding such polypeptides.

### CROSS-REFERENCE TO RELATED APPLICATIONS

This application is entitled to priority, pursuant to 35 U.S.C. § 119(e) to U.S. provisional patent application No. 60/186,141, filed Mar. 1, 2000.

### STATEMENT REGARDING FEDERALLY SUPPORTED RESEARCH OR DEVELOPMENT

This research was supported in part by U.S. Government funds (National Institute of Mental Health grant number 1RO1MH58324-02), and the U.S. Government may therefore have certain rights in the invention.

### BACKGROUND OF THE INVENTION

Glycogen synthase kinase-3 (GSK-3) is a serine/threonine protein kinase having a monomeric structure and a size of approximately 47 kilodaltons. It is one of several protein kinases which phosphorylate glycogen synthase (Embi, et al., 1980, Eur. J. Biochem., 107:519-527; Hemmings et al., 1982, Eur. J. Biochem. 119:443-451). GSK-3 is also referred to in the literature as factor A (F_{A}) in the context of its ability to phosphorylate F_{C}, a protein phosphatase (Vandenheede et al., 1980, J. Biol. Chem. 255:11768-11774). Other names for GSK-3 and homologs thereof include zeste-white3/shaggy (zw3/sgg; the Drosophila melanogaster homolog), ATP-citrate lyase kinase (ACLK or MFPK; Ramakrishna et al., 1989, Biochem. 28:856-860; Ramakrishna et al., 1985, J. Biol. Chem. 260:12280-12286), GSKA (the Dictyostelum homolog; Harwood et al., 1995, Cell 80:139-48), and MDSI, MCK1, and others (yeast homologs; Hunter et al., 1997, TIBS 22:18-22). GSK-3 beta has an essential role in protozoans such as Dictyostelum discoideum and Saccharomyces cerevisiae, in which it is required for sporulation (Harwood et al., 1995, Cell 80:139-148; Mitchell, 1994, Microbiol. Rev. 58:56-70).

The gene encoding GSK-3 is highly conserved across diverse phyla. In vertebrates, GSK-3 exists in two isoforms, designated GSK-3 alpha and GSK-3 beta. The amino acid identity among vertebrate homologs of GSK-3 is in excess of 98% within the catalytic domain (Plyte et al., 1992, Biochim. Biophys. Acta 1114:147-162), although GSK-3 alpha is known to be slightly larger than GSK-3 beta. It has been reported that there is only one form of GSK-3 in invertebrates, which appears to more closely resemble GSK-3 beta than GSK-3 alpha. Amino acid similarities (allowing for conservative replacements) between the slime mold and fission yeast proteins with the catalytic domain of human GSK-3 beta are 81% and 78%, respectively (Plyte et al., 1992, supra). The remarkably high degree of conservation across the phylogenetic spectrum suggests a fundamental role for GSK-3 in cellular processes.

GSK-3 phosphorylates numerous proteins in vitro, including beta-catenin, glycogen synthase, phosphatase inhibitor I-2, the type-II subunit of cAMP-dependent protein kinase, the G-subunit of phosphatase-1, ATP-citrate lyase, acetyl coenzyme A carboxylase, myelin basic protein, a microtubule-associated protein, a neurofilament protein, an N-CAM cell adhesion molecule, nerve growth factor receptor, c-Jun transcription factor, JunD transcription factor, c-Myb transcription factor, c-Myc transcription factor, L-myc transcription factor, adenomatous polyposis coli tumor suppressor protein, and tau protein (Plyte et al., 1992, Biochim. Biophys. Acta 1114:147-162; Korinek et al., 1997, Science 275:1784-1787; Miller et al., 1996, Genes & Dev. 10:2527-2539). The phosphorylation site recognized by GSK-3 has been determined in several of these proteins (Plyte et al., 1992, supra). The diversity of these proteins belies a wide role for GSK-3 in the control of cellular metabolism, growth, and development. GSK-3 tends to phosphorylate serine and threonine residues in a proline-rich environment, but does not display the absolute dependence upon these amino acids which is displayed by protein kinases which are members of the mitogen-activated protein (MAP) kinase or cdc2 families of kinases.

Among the proteins which are phosphorylated by GSK-3 is c-Jun, the expression product of the c-jun proto-oncogene and the cellular homolog of the v-jun oncogene of avian sarcoma virus (Dent et al., 1989, FEBS Lett. 248:67-72). Jun acts as a component of the activator protein-1 (AP-1) transcription factor complex, which binds to a palindromic consensus binding site (the AP-1 site). c-Jun is both necessary and sufficient to induce transcription of genes having an AP-1 site (Angel et al., 1988, Nature 332:166-171; Angel et al., 1988, Cell: 55:875-885; Chiu et al., 1988, Cell 54:541-552; Bohmann et al., 1989, Cell 59:709-717; Abate et al., 1990, Mol. Cell. Biol. 10:5532-5535). Transcription of a gene having an AP-1 site can be initiated by either a Fos-Jun heterodimer or by a Jun-Jun homodimer, although the Fos-Jun heterodimer binds to DNA more stably than the Jun-Jun homodimer and is consequently a more potent transcription activator. Fos is the expression product of another proto-oncogene, c-fos (Schonthal et al., 1988, Cell 54:325-334; Sassone-Corsi, 1988, Nature 334:314-319). Phosphorylation of c-Jun by GSK-3 significantly reduces the binding affinity of Jun-Jun homodimer for AP-1 sites (Boyle et al., 1991, Cell 64:573-584; Plyte et al., 1992, supra).

GSK-3 is a negative regulator of the wnt signaling pathway. The wnt pathway is a highly conserved signaling pathway that regulates cell fate decisions in both vertebrates and invertebrates (Perrimon, 1994, Cell 76:781-784; Perrimon, 1996, Cell 86:513-516; Miller et al., 1996, Genes & Dev. 10:2527-2539). Much of the pathway has been determined from detailed genetic analysis in Drosophila. At present, identified components of this signaling pathway include wnts (the secreted ligand), frizzled (the wnt receptor), and the intracellular mediators disheveled, GSK-3 (designated zw3/sgg in Drosophila), and beta-catenin (designated “armadillo” in Drosophila). In 10T½ cells, wnt signaling inhibits GSK-3 beta enzymatic activity (Cook et al., 1996, EMBO J. 15:4526-4536). This result is consistent with epistasis experiments in Drosophila which suggest that zeste white-3/GSK-3 beta functions downstream of disheveled and upstream of armadillo/beta-catenin. Wnt signaling leads to stabilization of beta-catenin protein in Drosophila (Peifer et al., 1994, Dev., 120:369-380; van Leeuwen, et al., 1994, Nature 368:342-344) as well as Xenopus (Yost et al., 1996, Genes & Dev., 10:1443-1454). It has also been demonstrated that treatment of Drosophila S2 cells with LiCl leads to accumulation of armadillo protein (Stambolic et al., 1996, Curr. Biol. 6:1664-1668). Stabilization of beta-catenin is associated with translocation of beta-catenin to the nuclei of cells responding to wnt signaling (Funayama et al., 1995, J. Cell Biol., 128:959-968; Schneider et al., 1996, Mech. Dev., 57:191-198; Yost et al., 1996, supra), binding between beta-catenin and LEF-1, and activation of transcription of wnt target genes (Cadigan et al., 1997, Genes Dev. 11:3286-3305; Miller et al., 1996, Genes Develop. 10:2527-2539). In addition, ectopic expression of conserved genes, including wnts, disheveled, and beta-catenin, leads to second axis formation in Xenopus. Second axis formation in Xenopus is also observed following lithium treatment. Although beta-catenin was originally discovered as a cadherin-binding protein, it has recently been shown to function as a transcriptional activator when complexed with members of the Tcf family of DNA binding proteins (Molenaar et al., 1996, Cell 86:391; Behrens et al., 1996, Nature 382:638). 100081 Recent data from several labs (Behrens et al., 1998, Science 280:596-599; Hart et al., 1998, Curr. Biol. 8:573-581; Hedgepeth et al., 1999, Mech. Develop. 80:147-151; Ikeda et al., 1998, EMBO J. 17:1371-1384; Itoh et al., 1998, Curr. Biol. 8:591-594; Sakanaka et al., 1998, Proc. Natl. Acad. Sci. USA 95:3020-3023) have demonstrated interaction of vertebrate GSK-3 beta with axin, the product of the fused locus in mice (Zeng et al., 1997, Cell 90:181-192). Mice homozygous for certain axin/fused alleles die at embryonic day 8-10 with ectopic dorsal axes and other developmental abnormalities (Gluecksohn-Schoenheimer, 1949, J. Exp. Zoology 110:47-76; Jacobs-Cohen et al., 1984, Genetic Res. 43:43-50). In addition, analysis in Xenopus embryos, using mouse axin (mAxin), indicates that axin can function as a negative regulator of the wnt pathway, since over-expression blocks endogenous dorsal development as well as dorsalization by ectopic wnt expression. Based on these observations, axin was proposed to be an inhibitor of dorsal axis formation (Zeng et al., 1997, Cell 90:181-192).

Molecular cloning of axin revealed that its gene encodes a protein with an amino terminal domain (designated an RGS domain) which exhibits sequence similarity to RGS proteins (which regulate heterotrimeric G-protein function). It has not yet been reported whether axin can regulate G-protein function. Axin also comprises a domain at its carboxyl terminus which exhibits amino acid sequence similarity to the protein encoded by the disheveled (DIX) locus. A Xenopus homologue of axin that is 69% identical to mammalian axin and also binds to GSK-3 beta has been identified (Hedgepeth et al., 1999, Mech. Develop. 80:147-151; GenBank Accession number AAC71036). Unlike murine axin, Xenopus axin exhibits remarkably high expression in the anterior midbrain during early development of the central nervous system and is ubiquitously expressed at a lower level.

Ventral expression of a dominant inhibitory mAxin mutant designated deltaRGS (i.e. a mutant lacking the RGS-like domain) in Xenopus causes dorsalization and axis duplication (Zeng et al., 1997, Cell 90:181-192). However, a deltaRGS mutant of human axin does not behave as a dominant negative in SW480 cells, but instead appears to facilitate the turnover of beta-catenin (Hart et al., 1998, Curr. Biol. 8:573-581). The mechanism by which the deltaRGS mutant exerts its dominant negative effects in Xenopus has not been studied. However, it has recently been reported that the tumor suppressor designated APC is able to bind to the RGS domain of Axin (Behrens et al., 1998, Science 280:596-599; Hart et al., 1998, Curr. Biol. 8:573-581; Kishida et al., 1998, J. Biol. Chem. 273:10823-10826), suggesting that the binding of APC to this region may be important for normal axis formation.

Recent data from several laboratories indicate that axin is part of a multimeric complex comprising GSK-3 beta, beta-catenin and APC (Hart et al., 1998, Curr. Biol. 8:573-581; Ikeda et al., 1998, EMBOJ. 17:1371-1384; Itoh et al., 1998, Curr. Biol. 8:591-594; Sakanaka et al., 1998, Proc. Natl. Acad. Sci. USA 95:3020-3023) which act together to regulate beta-catenin stability. Recent work indicates that axin interacts with protein phosphatase 2A, and that axin also interacts with itself (Hsu et al., 1999, J. Biol. Chem. 274 274:3439-3445). However, the functional significance of this self-interaction has not been elucidated. Axin binds with GSK-3 beta in vitro, in COS cells (Ikeda et al., 1998, EMBO J. 17:1371-1384), and in Xenopus (Itoh et al., 1998, Curr. Biol. 8:591-594). This binding facilitates phosphorylation of beta-catenin by GSK-3 beta in vitro (Ikeda et al., 1998, EMBO J. 17:1371-1384). Furthermore, over-expression of full length axin in SW480 cells increases beta-catenin turnover and blocks downstream TCF/LEF-1 mediated transcriptional activity (Hart et al., 1998, Curr. Biol. 8:573-581; Sakanaka et al., 1998, Proc. Natl. Acad. Sci. USA 95:3020-3023). The GSK-3 beta and beta-catenin binding sites of axin are located in close proximity to one another, suggesting that axin acts as a scaffold bringing the enzyme (GSK-3 beta) and its substrate ( beta-catenin) into close proximity (Ikeda et al., 1998, EMBO J.17:1371-1384). However, binding of GSK-3 beta with axin has not been shown to affect the enzymatic activity of GSK-3 beta.

In addition to axin, another GSK-3 beta binding protein (designated GBP) has been identified in Xenopus (Yost et al., 1998, Cell 93:1031-1041). In addition to binding GSK-3 beta, GBP inhibits GSK-3 beta activity in vivo. Furthermore, expression of GBP in ventral blastomeres of Xenopus embryos potently induces ectopic dorsal axis formation. Antisense depletion studies indicate that GBP is required for dorsal axis formation. The mechanism by which GBP regulates GSK-3 beta activity has not yet been elucidated.

The activity of GSK-3 beta is inhibited by lithium (Klein et al., 1996, Proc. Natl. Acad. Sci. USA 93:8455-8459; Hedgepeth et al., 1997, Dev. Biol. 185:82-91). Inhibition of GSK-3 beta is a physiological mechanism by which lithium exerts its therapeutic effects in animals (e.g. humans) afflicted with a variety of disorders. For example, lithium is an effective drug for treatment of bipolar (manic-depressive) disorder (Price et al., 1994, New Eng. J. Med. 331:591-598; Goodwin et al., 1990, In: Manic-Depressive Illness, New York: Oxford University Press). Lithium reduces the frequency and severity of recurrent episodes of mania and depression in patients with bipolar and unipolar disorders (Goodwin, et al., 1990, supra). Lithium can be used to treat profound depression in some cases. Despite the remarkable efficacy of lithium observed during decades of its use, the molecular mechanism(s) underlying its therapeutic actions have not been fully elucidated (Bunney, et al., 1987, In: Psychopharmacology: The Third Generation of Progress, Hy, ed., New York, Raven Press, 553-565; Jope et al., 1994, Biochem. Pharmacol. 47:429-441; Risby et al., 1991, Arch. Gen. Psychiatry 48:513-524; Wood et al., 1987, Psychol. Med. 17:570-600).

Lithium does not have an immediate effect during treatment of mania, but instead requires several weeks to manifest a clinical response. It has been suggested that this delay reflects changes in the expression of genes involved in alleviation of mania (Manji et al., 1995, Arch. Gen. Psychiatry 52:531-543).

In addition to its use as a therapeutic drug for the treatment of mania, lithium exhibits numerous other physiological effects in animals. For example, lithium mimics insulin action by stimulating glycogen synthesis (Bosch et al., 1986, J. Biol. Chem. 261:16927-16931). Further, exposure to lithium has dramatic morphogenic effects during the early development of numerous organisms. The effects of lithium on the development of diverse organisms, including Dictyostelum, sea urchins, zebrafish, and Xenopus have been reported (Maeda, 1970, Dev. Growth & Differ. 12:217-227; Van Lookeren Campagne et al., 1988, Dev. Genet. 9:589-596; Kao et al., 1986, Nature 322:371-373; Stachel et al., 1993, Development 117:1261-1274; Livingston et al., 1989. Proc. Natl. Acad. Sci. U.S.A. 86:3669-3673). In Dictyostelum discoideum, lithium alters cell fate by blocking spore cell development and promoting stalk cell development (Maeda, 1970, supra; Van Lookeren Campagne et al., 1988, supra). In Xenopus, lithium induces an expansion of dorsal mesoderm, leading to duplication of the dorsal axis or, in extreme cases, entirely dorsalized embryos which lack identifiably ventral tissues (Kao et al., 1986, Nature 322:371-373). Lithium also rescues UV-ventralized embryos (Kao et al., 1986, supra). In addition, treatment of sea urchin animal blastomeres with lithium induces the blastomeres to display a morphology resembling that of isolated vegetal blastomeres (Horstadius, 1973, In: Experimental Embryology of Echinoderms, Oxford University Press, Oxford).

Even though lithium is effective for the treatment of mania and other disorders in human patients, lithium treatment in humans is accompanied by several serious drawbacks (Baraban, 1994, Proc. Natl. Acad. Sci. U.S.A. 91:5738-5739). Particularly troublesome is the slim margin between therapeutic and toxic levels of lithium in vivo. Furthermore, because clearance of lithium is intimately tied to sodium and water excretion, a slight change in electrolyte balance can precipitate a life-threatening increase in lithium levels in vivo. In addition, even tight regulation of lithium within its therapeutic window is associated with a wide range of side effects, such as tremor, renal dysfunction, thyroid abnormalities, and birth defects (Jefferson et al., 1989, In: Comprehensive Textbook of Psychiatry, Kaplan et al., eds., Williams & Wilkins, Baltimore, vol. 2, 1655-1662). It is recommended that facilities for prompt and accurate serum lithium determinations be available before administering lithium to a patient (Physicians Desk Reference, 51st Ed., 1997, p. 2658). In addition, lithium should generally not be administered to patients having significant renal or cardiovascular disease, severe debilitation or dehydration, sodium depletion, or to patients receiving diuretics, since the risk of lithium toxicity is very high in such patients (Physicians Desk Reference, 51 st Ed., 1997, p.2352).

There exists a pressing need to identify compositions which have the therapeutic effect of lithium without the attendant side effects which accompany administration of lithium to human patients.

### BRIEF SUMMARY OF THE INVENTION

The invention relates to a composition that inhibits glycogen synthase kinase 3 beta (GSK-3 beta) activity, for example in vivo. The composition comprises a polypeptide of not more than about 60 amino acid residues, the polypeptide having an amino acid sequence which comprises the sequence:

Val-Xaa₅-Pro-Xaa₇-Xaag-Phe-Ala-Xaa₁₁-Glu-Leu-Ile-Xaa₁₅-Arg-Leu-Glu-Xaa₁₉-Xaa₂₀-Xaa₂₁-Xaa₂₂-Xaa₂₃-Xaa₂₄-Glu (SEQ ID NO: 9).

In this sequence,

each of Xaa₇, Xaa₈, Xaa₁₁, and Xaa₁₉ is independently any amino acid residue,

Xaa₅ is a negatively-charged amino acid residue,

Xaa₁₅ is a polar amino acid residue,

Xaa₂₀ is a non-polar aliphatic amino acid residue, and

at least two of Xaa₂₁, Xaa₂₂, Xaa₂₃, Xaa₂₄ are polar amino acid residues, the balance of Xaa₂₁, Xaa₂₂, Xaa₂₃, Xaa₂₄ being any amino acid residue.

In one embodiment, at least two of Xaa₂₁, Xaa₂₂, Xaa₂₃, Xaa₂₄ are charged amino acid residues. Preferably, Xaa₇ is a polar amino acid residue, Xaag can be Lys, Xaa₂₀ is Val, Xaa₂₁ is any amino acid residue, Xaa₂₂ is a positively-charged amino acid residue, Xaa₂₃ is a polar amino acid residue, and Xaa24 is Arg. For instance, the amino acid sequence can comprise the sequence

Xaa₁-Xaa₂-Xaa₃-Val-Xaa₅-Pro-Xaa₇-Xaa₈-Phe-Ala-Xaa₁₁-Glu-Leu-Ile-Xaa₁₅-Arg-Leu-Glu-Xaa₁₉-Xaa₂₀-Xaa₂₁-Xaa₂₂-Xaa₂₃-Xaa₂₄-Glu (SEQ ID NO: 10),

wherein each of Xaa₁, Xaa₂, and Xaa₃ is independently any amino acid residue. Species included within this embodiment include those wherein Xaa₁ is a negatively-charged amino acid residue, Xaa₂ is a non-polar amino acid residue, and Xaa₃ is a positively-charged amino acid residue. When the amino acid sequence comprises the sequence

Xaa₁-Xaa₂-Xaa₃-Val-Xaa₅-Pro-Xaa₇-Xaa₈-Phe-Ala-Xaa₁₁-Glu-Leu-Ile-Xaa₁₅-Arg-Leu-Glu-Xaa₁₉-Xaa₂₀-Xaa₂₁-Xaa₂₂-Xaa₂₃-Xaa₂₄-Glu (SEQ ID NO: 11)

(each of Xaa₁

, Xaa₂, and Xaa₃ independently being any amino acid residue), representative species include those in which

Xaa₁ is selected from the group consisting of Asp, Glu, and Met;

Xaa₂ is selected from the group consisting of Ile, Val, and Thr;

Xaa₃ is selected from the group consisting of His, Arg, and Pro;

Xaa₅ is selected from the group consisting of Asp and Glu;

Xaa₇ is selected from the group consisting of Glu, Gln, and Ala;

Xaa₈ is selected from the group consisting of Lys, Thr, and Ala;

Xaa₁₁ is selected from the group consisting of Ala and Glu;

Xaa₁₅ is selected from the group consisting of Ser, Asn, and His;

Xaa₁₉ is selected from the group consisting of Gly, Glu, Ala, and Lys;

Xaa₂₀ is selected from the group consisting of Val and Leu;

Xaa21 is selected from the group consisting of Leu, Gln, and Lys;

Xaa₂₂ is selected from the group consisting of Arg, Lys, and Leu;

Xaa₂₃ is selected from the group consisting of Asp, Glu, and Thr; and

Xaa₂₄ is selected from the group consisting of Arg and Leu.

Such species include those in which the sequence selected from the group consisting of SEQ ID NOs: 1-7, as well as those wherein

Xaa₁ is selected from the group consisting of Asp and Glu;

Xaa₂ is selected from the group consisting of Ile and Val;

Xaa₃ is selected from the group consisting of His and Arg;

Xaa₇ is selected from the group consisting of Glu and Gln;

Xaa₈ is Lys;

Xaa₁₉ is selected from the group consisting of Gly, Glu, and Ala;

Xaa₂₀ is Val;

Xaa₂₁ is selected from the group consisting of Leu and Gln;

Xaa₂₂ is selected from the group consisting of Arg and Lys; and

Xaa₂₄ is Arg (e.g., SEQ ID NOs: 1-4).

The polypeptide of the GSK-3 beta activity-inhibiting composition can be a polypeptide of less than about 30 amino acid residues. The composition can comprise a pharmaceutically acceptable carrier.

The invention also includes an antibody which binds specifically with the polypeptide of the GSK-3 beta activity-inhibiting composition, as well as a transgenic animal (e.g., a mouse) which comprises a transgene that encodes the polypeptide. In the transgenic animal, the portion of the transgene that encodes the polypeptide can, optionally, be operably linked with a controllable (e.g., inducible or tissue-specific) promoter.

The invention also includes a kit for inhibiting glycogen synthase kinase 3 beta activity in vivo. The kit comprises the GSK-3 beta activity-inhibiting composition described above and an instructional material. The instructional material can, for example, be one selected from the group consisting of an instructional material that describes administration of the composition to an animal in order to inhibit GSK-3 beta activity, an instructional material that describes administration of the composition to an animal in order to activate wnt signaling, an instructional material that describes administration of the composition to an animal in order to alleviate a disorder known to be alleviated by administration of lithium, and an instructional material that describes administration of the composition to a mammal in order to inhibit spermatozoal motility.

The invention further includes a method of inhibiting GSK-3 beta activity in vivo in an animal, the method comprising administering the GSK-3 beta activity-inhibiting composition described above to the animal. This same method can be used to activate wnt signaling in the animal or to alleviate a disorder known to be alleviated by administration of lithium to the animal. By way of example, such disorders include bipolar disorder, mania, depression, Alzheimer's disease, diabetes, and leukopenia.

The invention also includes a method of inhibiting motility of mammalian spernatozoa. This method comprises contacting the spermatozoa and the GSK-3 beta activity-inhibiting composition described above.

The invention further includes a method of inhibiting phosphorylation of a protein in a cell. The protein can, for example, be one selected from the group consisting of beta-catenin, glycogen synthase, phosphatase inhibitor I-2, the type-II subunit of cAMP-dependent protein kinase, the G-subunit of phosphatase-1, ATP-citrate lyase, acetyl coenzyme A carboxylase, myelin basic protein, a microtubule-associated protein, a neurofilament protein, an N-CAM cell adhesion molecule, nerve growth factor receptor, c-Jun transcription factor, JunD transcription factor, c-Myb transcription factor, c-Myc transcription factor, L-myc transcription factor, adenomatous polyposis coli tumor suppressor protein, and tau protein. The method comprises providing the GSK-3 beta activity-inhibiting composition described above to the cell.

In another aspect, the invention includes a composition that inhibits glycogen synthase kinase 3 beta activity in vivo. The composition comprises a polypeptide having the amino acid sequence of at least a portion of the region between the GID domain and the DIX domain of an axin. For example, the polypeptide can have the amino acid sequence of at least a portion of residues 489-777 of SEQ ID NO: 8, including the entirety of a residues 489-777 of SEQ ID NO: 8. This composition can be administered to an animal in order to alleviate a disorder known to be alleviated by administration of lithium in the animal.

In yet another aspect, the invention includes a method of identifying an in vivo inhibitor of GSK-3 beta activity. This method comprises assessing GSK-3 beta activity in an in vivo assay system in the presence and absence a polypeptide having an amino acid sequence that consists of less than all of the sequence between the GID domain and the DIX domain of an axin. The polypeptide is an inhibitor of glycogen synthase kinase 3 beta activity if the activity in the assay system is greater in the absence of the polypeptide than in the presence of the polypeptide. For example, the polypeptide can have an amino acid sequence that consists of less than all of residues 489-777 of SEQ ID NO: 8.

### BRIEF DESCRIPTION OF THE DRAWINGS

FIG. 1, comprising FIGS. 1A, 1B, and 1C, relates to inhibition of GSK-3 beta activity by the axin-GSK-3 beta interaction domain (GID).

FIG. 1A is a diagram of the myc-tagged constructs used in GSK-3 beta co-immunoprecipitation and activity assays described in Example 1. In FIG. 1A, full length (FL) Xaxin (amino acid residues 1-842) is indicated by the box at the bottom of the figure, in which the gray region represents the RGS domain, the solid region represents the GID domain, and the vertically striped region represents the DIX domain. Boxes corresponding to constructs designated “N-term” (i.e. amino acid residues 63-288 of Xaxin), “GID1” (i.e. amino acid residues 277-545 of Xaxin), and “C-Term” (i.e. amino acid residues 429-713 of axin) are shown above the portion of FL Xaxin to which the constructs correspond. The presence of all or part of a domain in a construct is indicated. The table at the right of FIG. 1A indicates if co-immunoprecipitation of GSK-3 beta and myc-tagged axin (“GSK binding”) or GSK-3 beta enzymatic activity (“GSK Activity”) was observed in Xenopus oocytes to which the corresponding construct was provided.

FIG. 1B is a set of images of immunoblots which indicate whether phosphorylated tau protein (“tau-P”) was formed in Xenopus oocytes to which tau protein (“tau”) was provided, when the oocytes expressed GSK-3 beta (lanes 2-6) and when FL Xaxin (lane 6) or the C-term (lane 3), GIDI (“GID”, lane 4), or N-term (lane 5) fragments of Xaxin were also provided to the oocytes. The image at the bottom of each of lanes 2-5 (“→gsk-3 beta”) is that of an immunoblot to detect GSK-3 beta protein.

FIG. 1C is a pair of images of immunoblots which indicate detection of GSK-3 beta protein which was co-immunoprecipitated with myc-tagged FL Xaxin or a myc-tagged Xaxin fragment.

FIG. 2, comprising FIGS. 2A, 2Bi-iv, and 2C, relates to activation of Wnt signaling in Xenopus embryos.

FIG. 2A is an image of an immunoblot of Xenopus cytoplasmic extracts obtained from Xenopus embryos in which beta-catenin was expressed, using an antibody which binds specifically with beta-catenin (described in McCrea et al., 1993, J. Cell. Biol. 123:477-84). The embryo corresponding to lane 2 was incubated in the presence of 20 millimolar LiCI. Extracts corresponding to lanes 3-5, 7, and 8 were obtained from embryos in which full-length (lane 8) Xaxin or the C-term (lane 3), GID1 (“GID”; lanes 4 and 7), or N-term (lane 5) fragments of Xaxin were expressed.

FIGS. 2Bi-iv are a quartet of images of Xenopus tadpoles in which axis duplication can be seen. The tadpoles in FIGS. 2Bi and 2Bii are shown at stage 40, and those in FIGS. 2Biii and 2Biv are shown at stage 30. The tadpoles in FIGS. 2Bii and 2Biv had been injected with 100 picograms of mRNA encoding the Xaxin GID into one ventral cell at the four cell embryo stage, and exhibit complete dorsal-anterior axis duplication. In FIG. 2Biv, the original axis is indicated by an arrow, and the secondary axis is indicated by an arrowhead.

FIG. 2C is a bar graph which dose dependence of axis duplication upon the amount of mRNA encoding the Xaxin GID1 (“GID”) fragment. GID mRNA was injected as above at the doses indicated in the figure and axis duplication was scored in tadpoles. Presence of cement gland and eyes was scored as complete axis duplication (shaded region of bars), and partial duplications of the trunk and/or heads lacking eyes or cement gland were scored as partial axis duplication (open region of bars).

FIG. 3, comprising FIGS. 3A, 3B, and 3C, relates to the structure of the GID of GSK-3 beta.

FIG. 3A is a diagram that depicts the structures of myc-tagged polypeptide constructs which were used to probe the structure of the GID. The constructs are depicted relative to their approximate positions in FL Xaxin and one another. Construct GID1 comprised amino acid residues 277-545 of Xaxin. Construct GID2 comprised amino acid residues 320-429 of Xaxin. Construct GID3 comprised amino acid residues 320-375 of Xaxin. Construct GID4 comprised amino acid residues 350-429 of Xaxin. Construct GID5 comprised amino acid residues 380-429 of Xaxin. Construct GID6 comprised amino acid residues 380-404 of Xaxin. The table on the right of the figure indicates whether each construct was co-immunoprecipitated (“GSK binding”) with GSK-3 beta and whether the construct inhibited (“inh”) GSK-3 beta activity (“GSK activity”; i.e. tau protein phosphorylating activity) when the construct was co-expressed in oocytes with GSK-3 beta.

FIG. 3B is an amino acid sequence alignment between the sequence of GID 6 (xAxin; SEQ ID NO: 1), and corresponding highly conserved sequences of chick axin (cAxin; SEQ IN NO: 2), murine axin (mAxin; SEQ ID NO: 3), and hunan axin (hAxin; SEQ ID NO: 4).

FIG. 3C further compares these four amino acid sequences with corresponding highly conserved sequences of three other axin-like proteins that have been reported, namely human axin 2 (hAxin2; SEQ ID NO: 5), rat axin (rAxil; SEQ ID NO: 6), and murine conductin (mConductin; SEQ ID NO: 7). In each of FIGS. 3B and 3C, similar amino acid residues are underlined, residues that are identical in each sequence are indicated in bold text, and a consensus sequence is listed beneath each group of sequences. Abbreviations used in the consensus sequences are: “x”, any amino acid residue; “-”, a negatively-charged amino acid residue; “+”, a positively-charged amino acid residue; “$”, a polar amino acid residue; “#”, a non-polar amino acid residue; “@”, a non-polar aliphatic amino acid residue; and “ˆ ”, a region in which at least two amino acid residues are polar, and preferably charged.

FIG. 4, comprising FIGS. 4A and 4B, indicate that the GID of Xaxin binds with GSK-3 beta, but does not inhibit GSK-3 beta activity in vitro.

FIG. 4A is an image of an immunoblot, made using an antibody which binds specifically with GSK-3 beta, of GSK-3 beta samples (“GSK”) which were incubated with purified his-tagged GID2 construct (“GID”) bound with nickel-agarose beads (“Ni Beads”), and then eluted from the beads. The relative amount of GID2 bound with the beads in lanes 4-6 is indicated by the wedge above the image.

FIG. 4B is a graph which depicts the relative activity (i.e. GS-2 peptide phosphorylating activity) of GSK-3 beta in the presence of selected concentrations of his-tagged GID2 construct (“GID/his”).

FIG. 5, comprising FIGS. 5A and 5B, relates to the AID of Xaxin.

FIG. 5A is a diagram that depicts the structures of GAL4 DNA binding domain-tagged polypeptide constructs which were used to probe the structure of the GID. The constructs were co-transformed into S. cerevisiae with FL Xaxin fused to the GAL4 activation domain in order to determine which constructs were able to bind with FL Xaxin. The constructs are depicted relative to their approximate positions in FL Xaxin and one another. Construct deltaDIX comprised amino acid residues 1-777 of Xaxin. Construct Y2H6 comprised amino acid residues 126-502 of Xaxin. Construct Y2H7 comprised amino acid residues 320-510 of Xaxin. Construct Y2H4 comprised amino acid residues 316-842 of Xaxin. Construct AID comprised amino acid residues 489-777 of Xaxin. The table on the right of the figure indicates whether each construct was capable of interacting with FL Xaxin.

FIG. 5B is a pair of images which depict immunoprecipitation of tagged axin proteins in Xenopus embryo cell extracts. Myc-tagged FL Xaxin (“myc-axin”), hemagglutinin epitope-tagged FL Xaxin (“HA-axin”), or both, were expressed in embryos. Samples were immunoprecipitated using an antibody which specifically binds with myc, and then immunoblotted using a labeled antibody which specifically binds with hemagglutinin epitope. The upper image (“myc-IP”) is an image of an immunoblot of embryo lysates prior to immunoprecipitation. The lower image (“lysates”) is an image of an immunoblot of immunoprecipitated samples.

FIG. 6, comprising FIGS. 6A and 6B, relates to the effect on GSK-3 beta binding and activity of deletion of the RGS, GID, or DIX domains of Xaxin.

FIG. 6A is a diagram that depicts the structures of myc-tagged polypeptide constructs which were used to probe the effects of these domains. The constructs were co-expressed in Xenopus oocytes with GSK-3 beta. The constructs are depicted relative to their approximately corresponding positions in FL Xaxin and one another. Construct GID2 comprised amino acid residues 320-429 of Xaxin. Construct deltaGID comprised amino acid residues 1-324 fused to residues 504-842 of Xaxin. Construct deltaRGS comprised amino acid residues 1-80 fused to residues 290-842 of Xaxin. Construct deltaDIX comprised amino acid residues 1-778 of Xaxin. The table at the right of the figure indicates whether each construct was capable of interacting with GSK-3 beta (“GSK binding”), as assessed by immunoprecipitation using an antibody which binds specifically with myc, followed by immunoblotting with an antibody which binds specifically with GSK-3 beta and whether each construct inhibited (“inh”) GSK-3 beta activity, as assessed by tau phosphorylation assay.

FIG. 6B is an image of an immunoblot of phosphorylated (“tau-P”) and non-phosphorylated (“tau”) tau protein, which exemplifies the dose-dependent effect of deltaRGS on GSK-3 beta activity. Wedges indicate relative amounts of GSK-3 beta (“GSK-3”) and construct deltaRGS co-expressed in Xenopus oocytes. Amounts of GSK-3 beta-encoding RNA were: for lanes 2 and 6, 20 nanograms; for lanes 3 and 7, 2 nanograms; for lanes 4 and 8, 1 nanogram; for lanes 5 and 9, 0.4 nanogram; and for lanes 10-13, 2 nanograms. Amounts of construct deltaRGS-encoding RNA were: for lanes 6-9, 20 nanograms; for lane 10, 20 nanograms; for lane 11, 2 nanograms; for lane 12, 1 nanogram; and for lane 13, 0.4 nanogram.

FIG. 7, comprising FIGS. 7A, 7Bi-vi, and 7C, relates to reversal of construct deltaRGS-induced dorsalization of Xenopus embryos by expression of construct deltaGID in the same embryos.

FIG. 7A is a diagram which depicts a proposed mechanism in which interaction (involving AID domains) between a construct deltaRGS polypeptide and a construct deltaGID polypeptide yields an axin dimer having a functional RGS domain and a functional GID domain.

FIGS. 7Bi and 7Biii are images which show complete axis duplication (i.e. eyes and cement glands present) in Xenopus embryos induced by expression of construct deltaRGS and construct GID 1, respectively. Axis duplication is reversed in embryos in which construct deltaGID and construct deltaRGS were co-expressed (FIG. 7Bii); however, axis duplication is not reversed in embryos in which constructs deltaGID and GID are co-expressed (FIG. 7Biv). Expression of construct deltaGID alone in embryos induces infrequent ectopic posterior axis formation (Figure Bvi). Embryos in FIG. 7Bv are controls.

FIG. 7C is a bar graph that in which scoring for complete and partial secondary axis formation is indicated for Xenopus embryos in which the indicated construct(s) were expressed. Shaded bars represent complete secondary axis formation (including eyes and cement glands), and open bars represent partial secondary axis formation (including head, but lacking eyes, cement gland, or both).

FIG. 8 is the amino acid sequence (SEQ ID NO: 8) of Xaxin.

FIG. 9 is an image of Western blots which demonstrate that the Xaxin GID stabilizes beta-catenin in neuro 2A cells which were transfected with a plasmid encoding the GID (“GID”) and maintained for 24 hours prior to blotting. The degree of beta-catenin stabilization was comparable to that obtained by treating control cells (“C”) with 20 millimolar LiCl (“Li+”). Antibodies used in the blot included those specific for beta-catenin (“β-catenin”), axin (“GID/axin”), and hnRNPK (“hnRNPK”; used as a loading control).

### DETAILED DESCRIPTION OF THE INVENTION

The present invention is based on the discovery within the amino acid sequence of an axin protein of an axin/GSK-3 beta interaction domain (GID) and a (separate) axin/axin interaction domain (AID). The GID is located between the RGS and DIX domains of axin, comprises as few as 22-25 amino acid residues, binds with GSK-3 beta both in cells, in a cellular milieu, or in vivo, and inhibits GSK-3 beta activity. Prior to the investigations described in this disclosure, it was not known that binding of axin with GSK-3 beta inhibits GSK-3 beta activity. The AID is located between the GID and DIX domains of axin, and facilitates binding between axin monomers. Prior to the investigations described in this disclosure, it was not known that interaction between axin monomers was necessary for formation of a complex that maintains the activity of GSK-3 beta and normal wnt signaling in vivo.

The invention includes polypeptides that inhibit GSK-3 beta activity and activate wnt signaling in vivo, as well as synthetic analogs of such polypeptides. These polypeptides have sequences derived from (including sequences identical to) the sequences of the GID and the AID of animal axins. The invention also includes methods of using such polypeptides to alleviate disorders related to aberrant GSK-3 beta activity or aberrant wnt signaling in animals such as humans. Because these polypeptides share a common activity with lithium (i.e. inhibiting GSK-3 beta activity), the polypeptides can also be used to alleviate disorders for which administration of lithium is a known treatment. The invention includes kits and compositions (e.g. pharmaceutical compositions) which comprise one or more of these polypeptides.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ ATTGCC 5′ and 3′ TATGCG 5′ share 50% homology. Any of a variety of known algorithms may be used to calculate the percent homology between two nucleic acids or two proteins of interest and these are well-known in the art.

An “isolated polypeptide” is a polypeptide which has been substantially separated from components (e.g., DNA, RNA, other proteins and peptides, carbohydrates and lipids) which naturally accompany it in a cell.

A disorder is “alleviated” if one or more of the frequency, the severity, and the duration of either the disorder or a symptom of the disorder are reduced.

The term “pharmaceutically acceptable carrier” means a chemical composition with which a pharmaceutically active agent can be combined and which, following the combination, can be used to administer the agent to a subject (e.g. a mammal such as a human).

The term “physiologically acceptable” ester or salt means an ester or salt form of a pharmaceutically active agent which is compatible with any other ingredients of the pharmaceutical composition and which is not deleterious to the subject to which the composition is to be administered.

Description

The invention relates, in one aspect, to a family of polypeptides, and synthetic analogs thereof, derived from the axin/GSK-3 beta interaction domain (GID) of axin proteins in animals. The inventors have discovered that these polypeptides can comprise as few as 22-25 amino acid residues, so long as those residues conform with a highly conserved region in the GID of animal axin proteins (hence these polypeptides are sometimes referred to herein as “GID-containing polypeptides”). Polypeptides of this sort can be of substantially any length (i.e. potentially including axin itself or axin less its RGS domain), but are preferably not more than about 60, 50, 45, 40, 35, or 30 amino acid residues in length. The amino acid sequence of each of these polypeptides comprises the following 22-residue sequence formula I.

Sequence Formula I

Val-Xaa₅-Pro-Xaa₇-Xaa₈-Phe-Ala-Xaa₁₁-Glu-Leu-Ile-Xaa₁₅-Arg-Leu-Glu-Xaa₁₉-Xaa₂₀-Xaa₂₁-Xaa₂₂-Xaa₂₃-Xaa24-Glu (SEQ ID NO: 9)

In sequence formula I:

1) each of Xaa₇, Xaa₈, Xaa₁₁, and Xaa₁₉ is independently any amino acid residue,

2) Xaa₅ is a negatively-charged amino acid residue,

3) Xaa₁₅ is a polar amino acid residue,

4) Xaa₂₀ is a non-polar aliphatic amino acid residue, and

5) at least two of Xaa₂₁, Xaa₂₂, Xaa₂₃, Xaa₂₄ are polar amino acid residues, the balance of Xaa₂₁, Xaa₂₂, Xaa₂₃, Xaa₂₄ any amino acid residue. Preferably, at least two of Xaa₂₁, Xaa₂₂, Xaa₂₃, Xaa₂₄ are charged amino acid residues.

In a preferred embodiment of sequence formula I, Xaa₇ is a polar amino acid residue, Xaa₈ is Lys, Xaa₂₀ is Val, Xaa₂₁ is any amino acid residue, Xaa₂₂ is a positively-charged amino acid residue, Xaa₂₃ is a polar amino acid residue, and Xaa₂₄ is Arg.

The amino acid sequence of a polypeptide, the sequence of which comprises sequence formula I, can alternatively be represented by the following sequence formula II.

Sequence Formula II

(Yaa)ₙ-Val-Xaa₅-Pro-Xaa₇-Xaa₈-Phe-Ala-Xaa₁₁-Glu-Leu-Ile-Xaa₁₅-Arg-Leu-Glu-Xaa₁₉-Xaa₂₀-Xaa21-Xaa₂₂-Xaa₂₃-Xaa₂₄-Glu-(Yaa)ₘ

In sequence formula II, each Yaa is independently any amino acid residue, n and m are positive integers having a sum not greater than about 28, 23, 18, 13, 8, or 3, and each Xaa has the meaning designated in sequence formula I.

A preferred polypeptide has a sequence comprising sequence formula III, which differs from sequence formula I in that three additional amino acid residues are specified at the amino terminus thereof

Sequence Formula III

Xaa₁-Xaa₂-Xaa₃-Val-Xaa₅-Pro-Xaa₇-Xaa₈-Phe-Ala-Xaa₁₁-Glu-Leu-Ile-XaalS-Arg-Leu-Glu-Xaa₁₉-Xaa₂₀-Xaa₂₁-Xaa₂₂-Xaa₂₃-Xaa₂₄-Glu (SEQ ID NO: 12)

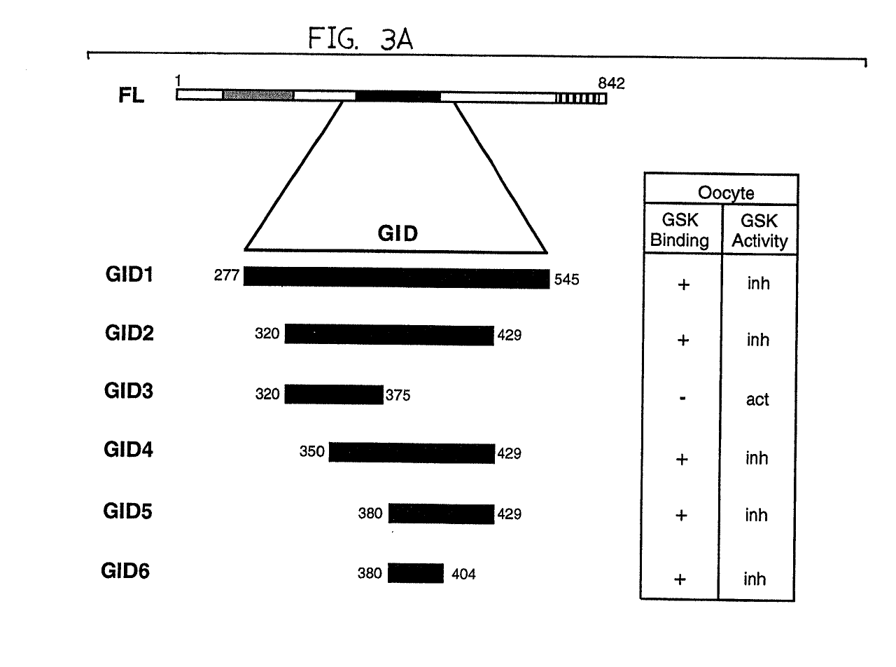

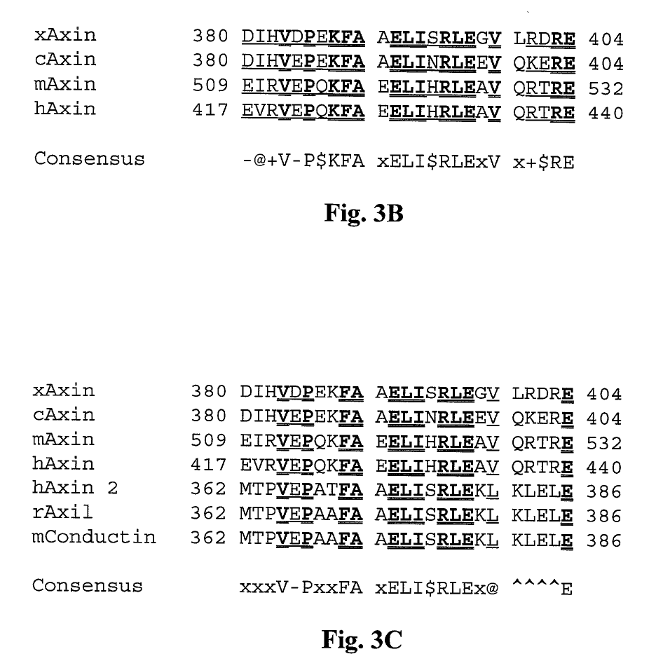

In sequence formula III, each of Xaa₁, Xaa₂, and Xaa₃ can be any amino acid residue. Preferably, however, Xaal is a negatively-charged amino acid residue, Xaa₂ is a non-polar amino acid residue, and Xaa₃ is a positively-charged amino acid residue. Sequence formula III can, for example, have the sequence of any one of SEQ ID NOs: 1-7 (as listed in FIG. 3C), and preferably has the sequence of one of SEQ ID NOs: 1-4.

In sequence formulas I-III each of the amino acid residues (when present in the formula) listed in the left column of Table I can, for example, be selected from the group of amino acid residues listed in the center portion of Table I, and each is preferably a residue listed in the right portion of Table I.

TABLE I

| Residue | Exemplary Residues | Preferred Residue(s) |
|---|---|---|
| Xaa₁ | Asp, Glu, Met | Asp, Glu |
| Xaa₂ | Ile, Val, Thr | Ile, Val |
| Xaa₃ | His, Arg, Pro | His, Arg |
| Xaa₅ | Asp, Glu | Asp, Glu |
| Xaa₇ | Glu, Gln, Ala | Glu, Gln |
| Xaa₈ | Lys, Thr, Ala | Lys |
| Xaa₁₁ | Ala, Glu | Ala, Glu |
| Xaa₁₅ | Ser, Asn, His | Ser, Asn, His |
| Xaa₁₉ | Gly, Glu, Ala, Lys | Gly, Glu, Ala |
| Xaa₂₀ | Val, Leu | Val |
| Xaa₂₁ | Leu, Gln, Lys | Leu, Gln |
| Xaa₂₂ | Arg, Lys, Leu | Arg, Lys |
| Xaa₂₃ | Asp, Glu, Thr | Asp, Glu, Thr |
| Xaa₂₄ | Arg, Leu | Arg |

The GID-containing polypeptides are preferably derived from naturally-occurring axin proteins. Such GID-containing polypeptides are preferably completely homologous to a portion of a naturally-occurring axin protein, the portion including a sequence corresponding to sequence formula I. However, such polypeptides need not be completely homologous to the portion. Instead, the polypeptides can exhibit 95%, 90%, 80%, 70%, 60%, or less sequence identity to the portion, particularly in the sequences not corresponding to sequence formula I. When the sequence of the polypeptide is derived from the sequence of a naturally-occurring axin protein, the polypeptide can be made by isolating the naturally-occurring axin and cleaving the non-desired portions therefrom, or it can be made using any of the other methods described herein or known in the art for synthesizing polypeptides.

As used herein, amino acid residues are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

Unless otherwise indicated, all amino acid sequences listed in this disclosure are listed in the order from the amino terminus to the carboxyl terminus.

The polypeptides described herein can be made, purified, or both, using any of a variety of techniques known in the art. Representative techniques include using an automated polypeptide synthesizing apparatus and recombinant techniques in which a nucleic acid encoding the polypeptide and operably linked with transcriptional and/or translational regulatory sequences (e.g. using any of a variety of known and commercially available expression vectors) is expressed to yield the polypeptide. Alternatively, a naturally-occurring axin protein can be isolated and cleaved to yield the polypeptide.

The present invention also provides analogs of polypeptides which bind with GSK-3 beta and inhibit the activity of this enzyme in vivo. Analogs can differ from peptides described herein by conservative amino acid sequence differences or by modifications which do not affect sequence, or by both.

For example, conservative amino acid changes may be made, which although they alter the primary sequence of the protein or peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or de-glycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Another family of polypeptides that is included in the invention is polypeptides that have the sequence of at least a portion (i.e. at least 20, 25, 30, 35, 40, 50, 75, 100, 150, 200, 250, or all 268 consecutive amino acid residues) of residues 489-777 of SEQ ID NO: 8 or the corresponding region of an animal analog (e.g. the corresponding region of human, murine, or rat axin, human axin 2, or murine conductin). In one aspect, the region corresponding to residues 489-777 of SEQ ID NO: 8 is the region of an axin protein sequence that lies between the GID and DIX domains of the sequence. These polypeptides include a functional AID (hence these polypeptides are sometimes referred to herein as “AID-containing polypeptides”), inhibit axin multimerization, inhibit the activity of GSK-3 beta in vivo, and activate wnt signaling. Thus, these polypeptides can be used for substantially all the same purposes described herein for GID-containing polypeptides (e.g. inhibition of GSK-3 beta activity, activation of wnt signaling, and treatment of disorders known to be alleviated by administration of lithium). The skilled artisan can determine, as described herein in the example, whether any particular polypeptide sequence obtained from the region between the GID and DIX domains of an axin with no more than ordinary experimentation.

The invention also includes antibodies (i.e., including antibodies of all classes, such as IgG, IgA, IgE, etc., single chain antibodies, and antibody fragments such as Fab and Fab₂ fragments) that bind specifically with the polypeptides described in this disclosure. These antibodies can bind with the GID domain of axin and thereby inhibit interaction between axin and GSK-3 beta. These antibodies can also be fixed to a substrate (e.g., the surface of an agarose or polyacrylamide gel or the surface of a chromatography particle) in order to immobilize axin or a polypeptide comprising a GID domain or analog thereof. Such substrates can be used, for example, for isolating or detecting the presence of axin or a GID-containing polypeptide.

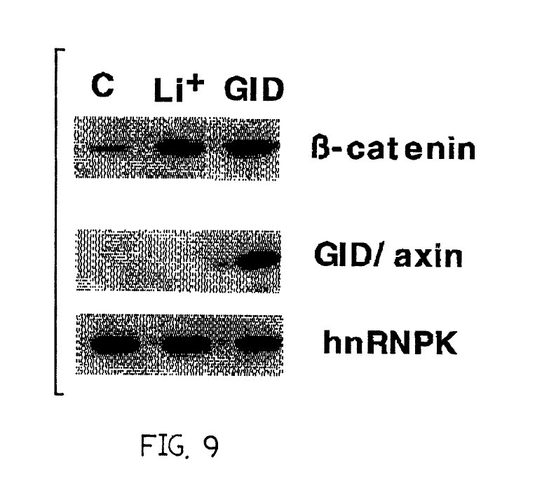

The polypeptides described in this disclosure can be incorporated into pharmaceutical compositions for ethical administration to humans and other animals. Such pharmaceutical compositions are described elsewhere herein. A pharmaceutical composition comprising a GID-containing polypeptide, an AID-containing polypeptide, or both, can be administered to an animal in order to inhibit GSK-3 (e.g. GSK-3 beta) activity in the animal, either partially or substantially completely. By inhibiting GSK-3 beta activity, phosphorylation of protein substrates of this enzyme can be inhibited. For example, administration of one of these pharmaceutical compositions to an animal can inhibit phosphorylation of one or more of beta-catenin, glycogen synthase, phosphatase inhibitor I-2, the type-II subunit of cAMP-dependent protein kinase, the G-subunit of phosphatase-1, ATP-citrate lyase, acetyl coenzyme A carboxylase, myelin basic protein, a microtubule-associated protein, a neurofilament protein, an N-CAM cell adhesion molecule, nerve growth factor receptor, c-Jun transcription factor, JunD transcription factor, c-Myb transcription factor, c-Myc transcription factor, L-myc transcription factor, adenomatous polyposis coli tumor suppressor protein, and tau protein. As a result, physiological processes associated with phosphorylation of the substrate protein can also be inhibited. By way of example, inhibition of beta-catenin phosphorylation effected by inhibiting GSK-3 beta activity in a mammal (e.g. a human) leads to cytoplasmic accumulation of (non-phosphorylated) beta-catenin, which leads to transport of beta-catenin into the nucleus, where it binds with LEF-1 and thereby activates expression of genes which are normally activated by wnt signaling. The results shown in FIG. 9 demonstrate that the GID of axin, when expressed from a plasmid in mammalian neuronal (neuro 2A) cells stabilizes beta-catenin to a degree comparable to that associated with lithium treatment of the cells.

Inhibition of GSK-3 beta activity also mimics the physiological effect of lithium administration, because lithium is also an inhibitor of GSK-3 beta. Thus, polypeptide inhibitors of GSK-3 beta, as described herein, can be used in place of lithium in human and veterinary therapy. Hunan disorders which are presently known to be treatable by administration of lithium include, for example, bipolar disorder, mania, depression, Alzheimer's disease, diabetes, and leukopenia. Each of these disorders can be alleviated by administering to a human afflicted with the disorder a composition comprising a GID-containing polypeptide or an AID-containing polypeptide described herein.

Another physiological effect exhibited by lithium, owing to its ability to inhibit the activity of GSK-3 beta, is that lithium inhibits the motility of mammalian spermatozoa. Because the GlD-containing polypeptides and AID-containing polypeptides inhibit GSK-3 beta activity, mammalian spermatozoal motility can be inhibited by contacting the spermatozoa with one or more of these polypeptides.

Pharmaceutical Compositions

The invention encompasses the preparation and use of medicaments and pharmaceutical compositions comprising a GID-containing polypeptide or an AID-containing polypeptide described herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. Administration of one of these pharmaceutical compositions to a subject is useful, for example, for alleviating disorders associated with aberrant GSK-3 beta activity or aberrant wnt signaling in the subject, as described elsewhere in the present disclosure. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys, fish including farm-raised fish and aquarium fish, and crustaceans such as farm-raised shellfish.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient. A unit dose of a pharmaceutical composition of the invention will generally comprise from about 1 nanogram to about 1 gram of the active ingredient, and preferably comprises from about 50 nanograms to about 10 milligrams of the active ingredient.

In addition to the active ingredient, a pharmaceutical composition of the invention may further comprise one or more additional pharmaceutically active agents. 25 Particularly contemplated additional agents include virus particles which comprise one or more polypeptides described herein or polynucleotide(s) encoding such a polypeptide. The polypeptides described herein can also be administered as fusion proteins, such as proteins which would facilitate entry into cells.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulfate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Oral compositions may be made, using known technology, which specifically release orally-administered agents in the small or large intestines of a human patient. For example, formulations for delivery to the gastrointestinal system, including the colon, include enteric coated systems, based, e.g., on methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. For example, a relatively thick polymer coating is used for delivery to the proximal colon (Hardy et al., 1987 Aliment. Pharmacol. Therap. 1:273-280). Polymers capable of providing site-specific colonic delivery can also be used, wherein the polymer relies on the bacterial flora of the large bowel to provide enzymatic degradation of the polymer coat and hence release of the drug. For example, azopolymers (U.S. Pat. No. 4,663,308), glycosides (Friend et al., 1984, J. Med. Chem. 27:261-268) and a variety of naturally available and modified polysaccharides (PCT GB 89/00581) may be used in such formulations.

Pulsed release technology such as that described in U.S. Pat. No. 4,777,049 may also be used to administer the active agent to a specific location within the gastrointestinal tract Such systems permit drug delivery at a predetermined time and can be used to deliver the active agent, optionally together with other additives that my alter the local microenvironment to promote agent stability and uptake, directly to the colon, without relying on external conditions other than the presence of water to provide in vivo release.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intravenous, intraarterial, intramuscular, or intrastemal injection and intravenous, intraarterial, or kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules, in multi-dose containers containing a preservative, or in single-use devices for auto-injection or injection by a medical practitioner. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

A pharmaceutical composition of the invention may be administered to deliver a dose of between 500 picograms per kilogram body weight per day and 1 milligrams per kilogram body weight per day to a subject.

It is understood that the ordinarily skilled physician or veterinarian will readily determine and prescribe an effective amount of the compound to alleviate a disorder associated with aberrant GSK-3 beta activity or aberrant wnt signaling in the subject. In so proceeding, the physician or veterinarian may, for example, prescribe a relatively low dose at first, subsequently increasing the dose until an appropriate response is obtained. It is further understood, however, that the specific dose level for any particular subject will depend upon a variety of factors including the activity of the specific compound employed, the age, body weight, general health, gender, and diet of the subject, the time of administration, the route of administration, the rate of excretion, any drug combination, and the severity of the disorder being treated.

Kits

Another aspect of the invention relates to a kit comprising a pharmaceutical composition of the invention and an instructional material. As used herein, an “instructional material” includes a publication, a recording, a diagram, or any other medium of expression which is used to communicate the usefulness of the pharmaceutical composition of the invention for inhibiting GSK-3 beta activity or activating wnt signaling in a subject. The instructional material may also, for example, describe an appropriate dose of the pharmaceutical composition of the invention. The instructional material of the kit of the invention may, for example, be affixed to a container which contains a pharmaceutical composition of the invention or be shipped together with a container which contains the pharmaceutical composition. Alternatively, the instructional material may be shipped separately from the container with the intention that the instructional material and the pharmaceutical composition be used cooperatively by the recipient.

The invention also includes a kit comprising a pharmaceutical composition of the invention and a delivery device for delivering the composition to a subject. By way of example, the delivery device may be a squeezable spray bottle, a metered-dose spray bottle, an aerosol spray device, an atomizer, a dry powder delivery device, a self-propelling solvent/powder-dispensing device, a syringe, a needle, a tampon, or a dosage measuring container. The kit may further comprise an instructional material as described herein. Transgenic Animals

The invention includes transgenic (preferably non-human) animals which comprise a transgene encoding a polypeptide described in this disclosure (i.e., a polypeptide having a sequence comprising or consisting of one of Sequence formulas I, II, and III). Expression of the transgene in the animal results in production of a polypeptide which is the GID of Xenopus axin, an analog of the Xenopus axin GID, the GID of axin of another species, or an analog of such a GID. The polypeptide is able to interact with GSK-3 beta, thereby preventing or inhibiting normal association of axin dimers (or other proteins) with GSK-3 beta. As a result, the activity of GSK-3 beta is inhibited, and the Wnt signaling pathway is activated. As disclosed elsewhere in this disclosure, lithium activates Wnt signaling. Thus, expression of the transgene can mimic the effect of lithium administration in the animal. The transgene preferably comprises a promoter from which initiation of transcription can be controlled. Numerous examples of controllable promoters are known in the art, and include inducible promoters, repressible promoters, temperature-sensitive promoters, and tissue-specific promoters. A preferred promoter is the calcium-calmodulin dependent protein kinase II alpha (CaMKIIalpha) promoter. Expression of polypeptide s operably linked with this promoter sequence is generally limited to adult neurons of the forebrain, including neurons of the neocortex, the hypothalamus, the amygdala, and the basal ganglia. The transgenic animal can be of any species for which transgenic generation methods are known (i.e., including at least mammals such as cows, goats, pigs, sheep, and rodents such as rats and mice).

The invention is now described with reference to the following Examples. This Examples are provided for the purpose of illustration only, and the invention is not limited to these Examples, but rather encompasses all variations which are evident as a result of the teaching provided herein.

### EXAMPLE 1

Regulation of Glycogen Synthase Kinase-3 Beta and Downstream Wnt Signaling by Axin

Axin is a protein encoded by the fused locus in mice that is required for normal vertebrate axis formation. The experiments presented in this example define a 25 amino acid residue portion of axin that comprises the glycogen synthase kinase-3 beta (GSK-3 beta) interaction domain (GID). In contrast to full length axin, which antagonizes Wnt signaling, the isolated 25-residue GID-containing polypeptide inhibits GSK-3 beta activity in vivo and activates Wnt signaling. Similarly, mutants of axin protein which lack key regulatory domains such as the RGS domain (which is required for interaction with the adenomatous polyposis coli {APC} protein) bind with GSK-3 beta protein and inhibit GSK-3 beta activity in vivo, suggesting that these domains are critical for proper regulation of GSK-3 beta activity. The experiments presented in this example also define a self-interaction domain within axin. Formation of an axin regulatory complex in vivo is critical for axis formation and GSK-3 beta activity. The results of the experiments presented in this example indicate that the axin complex can directly regulate GSK-3 beta activity in vivo. These results also demonstrate that inhibitors of GSK-3 beta can mimic the effect of lithium in developing Xenopus embryos and in other biological systems.

The materials and methods used in the experiments presented in this example are now described.

Recombinantly-produced GSK-3 beta protein was purchased from New England Biolabs (Beverly, Mass.). Gamma P-ATP was obtained from Amersham (Arlington Heights, Ill.). Western analysis was performed using enhanced chemiluminescence reagents, also obtained from Amersham.

DNA Constructs

DNA fragments corresponding to a portion of Xenopus axin (Xaxin) near the amino terminal end (designated “N-term” and encoding amino acid residues 63-288), a central portion (designated “GID-1 ” and encoding amino acid residues 277-545), and a portion near the carboxyl terminal end (designated “C-term” and encoding residues 429-713) were isolated from a stage VI Xenopus oocyte cDNA library as described (Hedgepeth et al., 1999, Mech. Develop. 80:147-151). These DNA fragments were sub-cloned into plasmid pCS2MT in frame with an N-terminal six-Myc epitope tag. Full length (FL) Xaxin was assembled into plasmid CS2MT using restriction fragments of partial cDNA clones, as well as PCR products, and the complete sequence was confirmed by DNA sequencing. The deletion constructs designated “GID-2” (i.e. encoding Xaxin amino acid residues 320-429), “GID-3” (i.e. encoding Xaxin amino acid residues 320-375), “GID-4” (i.e. encoding Xaxin amino acid residues 350-429), “GID-5” (i.e. encoding Xaxin amino acid residues 380-429), “GID-6” (i.e. encoding Xaxin amino acid residues 380-404), “deltaGID” (i.e. encoding Xaxin having amino acid residues 324-504 deleted therefrom), and “deltaRGS” (i.e. encoding Xaxin having amino acid residues 80-290 deleted therefrom) were cloned in-frame following the Myc tag of pCS2MT using PCR products generated from the FL Xaxin template. deltaDIX (i.e. encoding Xaxin having amino acid residues 778-842 deleted therefrom) was created by restriction endonuclease digestion of FL Xaxin in pCS2MT. The polypeptide encoded by a GID-1 DNA construct lacking the Myc-epitope tag had similar activity to the Myc-tagged construct.

Xenopus Embryo and Oocyte Expression

Stage VI Xenopus oocytes were isolated by collagenase treatment (Smith et al., 1994, Meth. Cell Biol. 36:45-58) and were injected with mRNA prepared by in vitro transcription (mMessage Machine™; Ambion, Austin, Tex.). 10 nanoliters of mRNA (containing 1-2 nanograms per nanoliter) was injected for each construct (unless otherwise specified), and the injected oocytes were incubated for 16 hours at 18° C. To analyze the effects of Xaxin constructs on dorsal-ventral pattern formation, Xenopus embryos were injected with 10 nanoliters of mRNA (0.1-0.2 nanogram per nanoliter) into one dorsal or ventral cell of a 4 cell embryo and dorsal axes were assessed at the tadpole stage. For Xaxin co-immunoprecipitation, fertilized eggs were injected with 10 nanoliters of Xaxin tagged with Myc, with hemagglutinin, or with both Myc and hemagglutinin (1 nanogram per nanoliter) and harvested at the blastula stage (stage 8).

GSK-3 Beta Assays

In ovo phosphorylation of tau by GSK-3 beta was performed by microinjection of tau protein into oocytes expressing GSK-3 beta and Xaxin constructs. After 90 minutes, oocytes were homogenized and tau phosphorylation was analyzed in western blots using phospho-specific antibodies as described (Hedgepeth et al., 1997, Develop. Biol. 185:82-91). In vitro assays for GSK-3 beta activity were performed as described (Klein et al., 1996, Proc. Natl. Acad. Sci. USA 93:8455-8459). Recombinant GID-2/his protein (described below) was purified by affinity chromatography and added to in vitro assays at selected concentrations.

Immunoprecipitation and Immunoblotting

Oocytes were homogenized in Triton X-100 lysis buffer (described in Rubinfeld et al., 1996, Science 272:1023-1026). Embryos expressing Xaxin tagged with Myc, with hemagglutinin, or with both, were homogenized in about 10 microliters per oocyte of a solution comprising 20 millimolar Tris, pH 7.6, 150 millimolar NaCi, 0.5% Triton X-100, 1 millimolar EDTA, 50 millimolar NaF, 0.5 millimolar NaVO4, 10 nanomolar microcystin, and Sigma bacterial protease inhibitor cocktail at 1:100. Lysates were immunoprecipitated using anti-Myc epitope antibodies (designated “9E10”) at a concentration of approximately 10 micrograms per milliliter. After 1 hour of incubation at 0° C., immune complexes were collected using anti-mouse-IgG coupled protein-A beads (Upstate Biotechnology, Lake Placid, N.Y.). The beads were washed three times using cold (0-4° C.) phosphate-buffered saline (PBS), and the complexes were eluted from the beads using Laemmli sample buffer. Eluted samples were separated on 10% (w/v) SDS-polyacrylamide gels, and the separated proteins were immunoblotted using anti-GSK-3 beta antibodies (0.25 microgram per milliliter; Transduction Labs, Lexington, Ky.), 9E 10 (anti-Myc) antibodies (1 microgram per milliliter), or anti-hemagglutinin antibodies (1 :1000; Amersham, Arlington Heights, Ill.). Antibody-hybridized proteins were visualized by chemiluminescent detection.

Yeast Two Hybrid Assay

FL Xaxin was cloned in frame with the GAL4 DNA binding domain (BD) in plasmid pAS2-1 (Clontech) to yield BD plasmid. Expression of BD plasmid yielded a Xaxin-GAL4 fusion protein. FL Xaxin, deltaDIX Xaxin, and a fragment (designated “AID”) encoding amino acid residues 489-777 of Xaxin were cloned in frame with the GAL4 transcriptional activation domain (AD) in the vector pACT2 to yield AD plasmids. Yeast were transformed with BD and AD plasmids using previously described protocols (Clontech, Palo Alto, Calif.). Transformed colonies were selected and assayed for beta-galactosidase activity in order to detect interacting proteins. In addition, three Xenopus axin partial length cDNAs (designated Y2H 2, Y2H 6, Y2H 7; all in plasmid pACT2) isolated from a previous yeast two hybrid screen (Hedgepeth et al., 1999, Mech. Develop. 80:147-151) were analyzed. Purification of GID

A cDNA (GID-2) encoding amino acid residues 320-429 of Xaxin was cloned into pET29b (Novagen, Madison, Wis.) in frame with the His-epitope tag. The resulting encoded GID-2/his fusion protein was expressed in BL2I/DE3 cells and purified on nickel agarose according to standard procedures. Purified GID-2/his was added to a reaction cocktail having a final volume of 20 microliters and containing 25 nanomolar recombinantly-generated GSK-3 beta in GSK-3 assay buffer, as described (Klein et al., 1996, Proc. Natl. Acad. Sci. USA 93:8455-8459). This reaction mixture was incubated at 0° C. for one hour. After this incubation period, half of the mixture was assayed for GSK-3 beta activity, and the other half was incubated with nickel-agarose at 4° C. for one hour with rotation. The agarose was washed three times using PBS, and then Laemmli sample buffer was added. The samples were boiled for five minutes and then subjected to immunoblotting using anti-GSK-3 beta antibody.

The results of the experiments presented in this example are now described.

The GSK-3 Beta Interaction Domain of Axin Potently Inhibits GSK-3 Beta Activity.

Xenopus axin protein was identified in a yeast two-hybrid screen using GSK-3 beta as an endogenous protein that might regulate GSK-3 beta activity (Hedgepeth et al., 1999, Mech. Develop. 80:147-151). That work was similar to the work of others who identified chick and mammalian axins (Behrens et al., 1998, Science 280:596-599; Hart et al., 1998, Curr. Biol. 8:573-581; Ikeda et al., 1998, EMBO J. 17:1371-1384; Itoh et al., 1998, Curr. Biol. 8:591-594; Sakanaka et al., 1998, Proc. Natl. Acad. Sci. USA 95:3020-3023). It was recognized that the interaction between axin and GSK-3 beta could potentially indicate that axin regulates GSK-3 beta activity directly. Although axin was known to act as a protein scaffold to bring the substrate beta-catenin within proximity to GSK-3 beta, direct regulation of GSK-3 beta enzymatic activity by axin had not previously been reported.

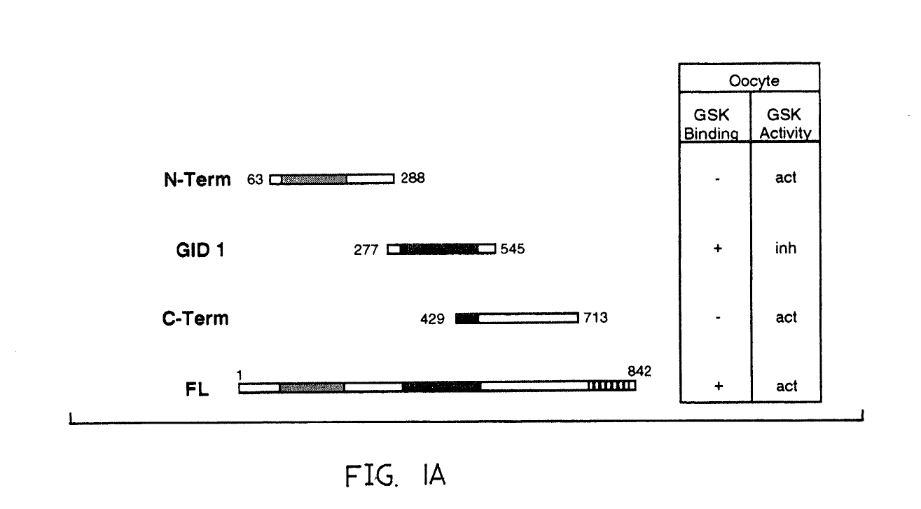

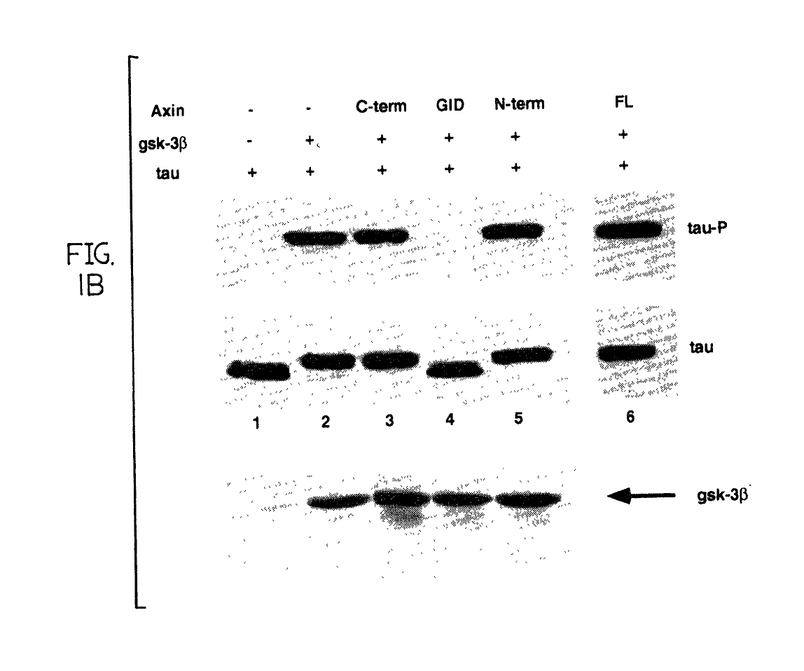

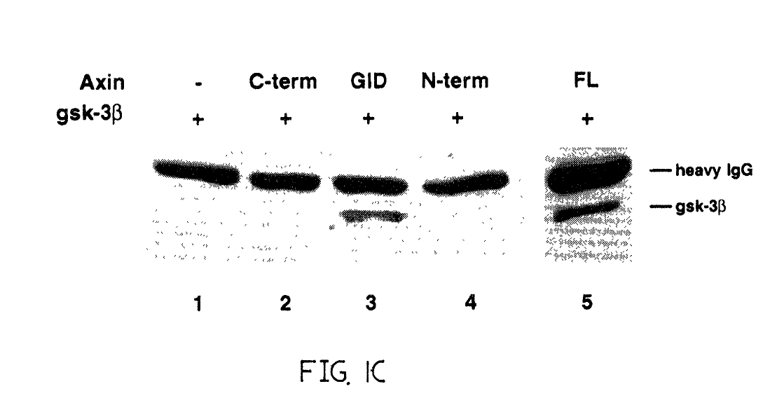

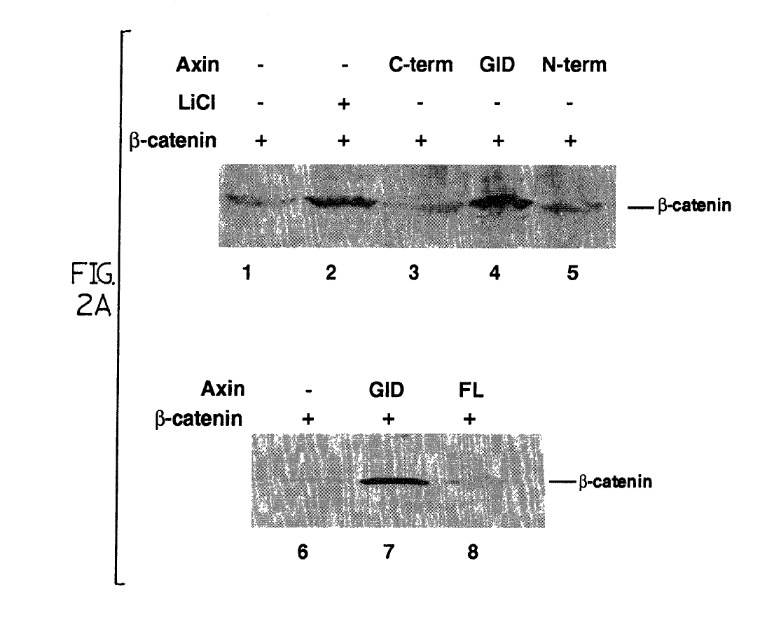

In the experiments presented in this example, a tau phosphorylation assay (described in Hedgepeth et al., 1997, Develop. Biol. 185:82-91) was used to examine the activity of GSK-3 beta in Xenopus oocytes in the presence of full length axin and axin deletion mutants. In this assay, phosphorylation of tau (detected by Western blotting using the phospho-specific tau antibody PHF-1; Greenberg et al., 1992, J. Biol. Chem. 267:564-569; Otvos et al., 1994, J. Neurosci. Res. 39:669-673), is completely dependent on expression of GSK-3 beta, as demonstrated previously (Hedgepeth et al., 1997, Develop. Biol. 185:82-91) and as indicated in FIG. 1B (compare lanes 1 and 2). Furthermore, GSK-3 beta-dependent tau phosphorylation occurs in oocytes at the same sites (tau serine residues 396 and 404) that are phosphorylated by GSK-3 beta in vitro and occurs with similarly rapid kinetics. These results confirm that PHF-1 immunoreactivity reflects GSK-3 beta activity, as described previously (Hedgepeth et al., 1997, Develop. Biol. 185:82-91).

GSK-3 beta was expressed in oocytes together with Myc-tagged full length Xaxin or with the amino-terminal (amino acid residues 63-288), GID-1 (amino acid residues 277-545), or carboxyl-terminal (amino acid residues 429-713) fragments of Xaxin, as depicted in FIG. 1A. Purified tau protein was then microinjected into the oocytes, and phosphorylation of tau was measured by immunoblotting using antibody PHF-1 or with antibodies that detect all (i.e. phosphorylated and non-phosphorylated) forms of tau.

Surprisingly, expression of the GID-1 fragment of Xaxin in the oocytes resulted in virtually complete inhibition of GSK-3 beta-mediated tau phosphorylation (as indicated in FIG. 1B, lane 4). This is evident from loss of PHF-1 immunoreactivity as well as from an increase in the electrophoretic mobility of tau protein (i.e. detected using phosphorylation-independent tau-specific antibodies). In contrast, expression of full length Xaxin in the oocytes (corresponding to lane 6 in FIG. 1B) had no discernible effect on tau phosphorylation. Inhibition of GSK-3 beta activity was not attributable to changes in the level of GSK-3 beta protein in the respective oocytes, as indicated by the results of Western detection of GSK-3 beta in the oocytes (FIG. 1B, lower panel, lanes 1-5). Co-expression of GSK-3 beta with either the amino- or carboxyl-terminal fragments of Xaxin (as indicated in lanes 3 and 5 of FIG. 1B), or with unrelated mRNAs, had no effect on GSK-3 beta activity. Both the GID-1 fragment and full length Xaxin bound with GSK-3 beta, as detected by immunoprecipitation of Myc-tagged axin constructs and Western blotting using a GSK-3 beta-specific antibody (as indicated in lanes 3 and 5 of FIG. 1C). Neither the amino- nor carboxyl-terminal fragments of axin bound with GSK-3 beta (as indicated in lanes 2 and 4 of FIG. 1C). These data indicate that binding of the Xaxin GID with GSK-3 beta inhibits the tau-phosphorylating enzymatic activity of GSK-3 beta.

The GID of Axin Activates Wnt Signaling

Although endogenous GSK-3 beta is not present at sufficient levels to detect using the tau assay, inhibition of GSK-3 beta is known to cause stabilization of cytoplasmic beta-catenin (Cadigan et al., 1997, Genes Dev. 11:3286-3305; Miller et al., 1996, Genes Develop. 10:2527-2539), and this serves as a widely used assay for downstream activation of wnt signaling. Inhibition of endogenous GSK-3 beta activity also causes stabilization of beta-catenin in Xenopus embryos and oocytes (Hedgepeth et al., 1997, Develop. Biol. 185:82-91; Yost et al., 1998, Cell 93:1031-1041; Yost et al., 1996, Genes Develop. 10:1443-1454). For this reason, levels of beta-catenin protein were assessed in Xenopus oocytes which had been injected either with full length Xaxin mRNA or with mRNA encoding one of the amino-terminal, GID, or carboxyl-terminal domains of Xaxin.

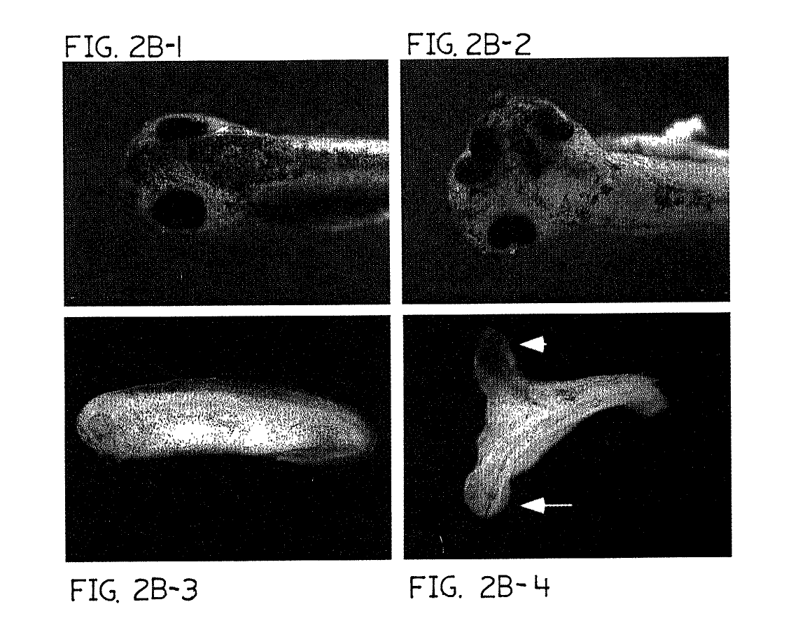

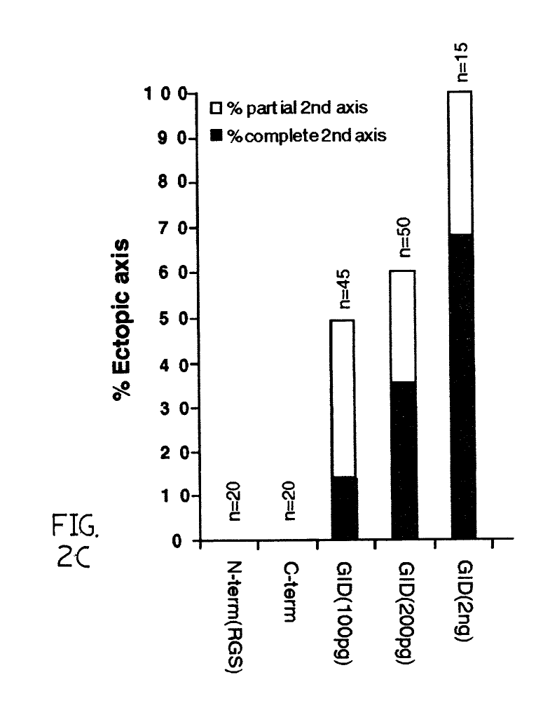

Expression of the GID in the Xenopus oocytes (as in FIG. 2A, lanes 4 and 7) resulted in accumulation of beta-catenin in the oocytes, an effect similar to that of treatment of Xenopus oocytes with lithium (as in FIG. 2A, lane 2), which is known to be a direct inhibitor of GSK-3 beta activity (Klein et al., 1996, Proc. Natl. Acad. Sci. USA 93:8455-8459; Stambolic et al., 1996, Curr. Biol. 6:1664-1668). Accumulation of beta-catenin was not observed in oocytes in which full length axin (FIG. 2A, lane 8) or either of the amino- or carboxyl-terminal domains of Xaxin was expressed (FIG. 2A, lanes 3 and 5, respectively). These observations, considered together with the observation described above that the GID of Xaxin inhibits tau phosphorylation mediated by GSK-3 beta, indicates that the GID activates downstream wnt signaling by inhibiting GSK-3 beta activity.

LEF-1 (also designated tcf) is a DNA-binding protein that also binds with beta-catenin. The beta-catenin/LEF-1 complex is known to activate transcription of wnt target genes. A genetic construct was made which comprised this LEF-1-binding promoter operably linked with a luciferase gene, for use as a reporter of wnt signaling. Using this construct, it was demonstrated in 293T cells that the axin GID, but not full length axin, strongly activated wnt signaling, demonstrating that the axin GID mimics the ability of lithium and wnts to activate LEF-1 dependent transcription.

Activation of wnt signaling in vivo has been shown by others to lead to axis duplication in Xenopus and mouse embryos (Heasman, J. 1997, Development 124:4179-4191; McMahon et al., 1989, Cell 58:1075-1084; Miller et al., 1996, Genes Develop. 10:2527-2539; Popperl et al., 1997, Development 124:2997-3005). It was hypothesized that, because inhibition of GSK-3 beta by the Xaxin GID apparently activates downstream Wnt signaling, expression of the GID on the ventral side of early Xenopus embryos would lead to axis duplication, similar to that observed following ventral expression of Wnts or dominant negative GSK-3 beta. In order to test this hypothesis, mRNA encoding full length Xaxin or the GID of Xaxin was microinjected into either ventral or dorsal blastomeres of 4 cell Xenopus embryos. The embryos were cultured until the tadpole stage, and the frequency of ectopic dorsal axes was scored.

Ventral injection of GID mRNA caused a high frequency of secondary axis formation, as shown in FIG. 2B. Complete axes, including eyes and cement gland, were induced in up to 65% of injected embryos, as indicated in FIG. 2C. Ectopic axes were detected when as little as 100 picograms of mRNA per embryo was microinjected. Dorsal injection of mRNA encoding the Xaxin GID had no effect on axial development. Conversely, full length axin caused ventralization when expressed in dorsal blastomeres, as described for mouse axin (Zeng et al., 1997, Cell 90:181-192) and induced formation of ectopic cement glands when expressed ventrally, as described for over-expression of GSK-3 beta (Itoh et al., 1995, Dev Suppl 121:3979-3988). In addition, injection of mRNA encoding either of the amino- or carboxyl-terminal domains of Xaxin had no apparent effect on axial development. These observations demonstrate that the GID activates downstream wnt signaling in Xenopus embryos, most likely by inhibiting GSK-3 beta activity and consequently stabilizing beta-catenin.

The ability of the Xaxin deletion mutants which include the GID to inhibit GSK-3 beta-mediated phosphorylation of tau protein, to induce accumulation of beta-catenin protein, and to induce ectopic axis formation in Xenopus oocytes, as described in this example, indicates that the GID of Xaxin binds with and inhibits GSK-3 beta and activates Wnt signaling.

A 25 amino acid residue portion of Xaxin is sufficient to bind and inhibit GSK-3 beta in vivo.

In order to identify the domain of axin necessary for GSK-3 beta binding, multiple Myc-tagged GID deletion constructs (shown diagrammatically in FIG. 3A) were individually expressed in Xenopus oocytes in which GSK-3 beta was also expressed. Tau protein was microinjected into the oocytes, and immunoblotting was performed using phosphorylation-state-specific tau antibodies. The ability of the each deletion construct to inhibit of GSK-3 beta activity is indicated in FIG. 3A. A parallel group of oocytes expressing GSK-3 beta and the Myc-tagged GID deletion constructs were lysed, and GID proteins were immunoprecipitated using a Myc-specific antibody. The presence or absence of GSK-3 beta in GID immunoprecipitates was detected using GSK-3 beta-specific antibodies. GID deletion constructs which included amino acid residues 380-404 of Xaxin (i.e. constructs GID-1, GID-2, GID-4, GID-5, and GID-6) bound GSK-3 beta and inhibited GSK-3 beta mediated tau phosphorylation, as indicated in FIG. 3A. GID-3, which lacks this 25 amino acid residue portion, did not bind GSK-3 beta and had no effect on GSK-3 beta activity. These data indicate that both the GSK-3 beta-binding and GSK-3 beta-inhibiting effects of the GID are mediated by this 25-amino acid residue portion. The amino acid sequence of this region is well conserved among Xenopus, chicken, mouse, and human axins, as indicated in FIG. 3B. Changing phenylalanine residue 388 in FIG. 3B to tyrosine reduces, but does not abolish, the activity of the polypeptide. However, changing leucine residue 392 in FIG. 3B to proline abolishes its activity.

As described in this example, the domain consisting of amino residues 380 to 404 of Xaxin is sufficient to bind with and inhibit GSK-3 beta and to activate Wnt signaling. The experiments described in this example also demonstrate that polypeptides comprising the GID and further comprising additional residues one or both ends of the GID (e.g. the polypeptides designated GID1, GID2, GID4, and GID5 in this example, but not full length Xaxin) also exhibit these activities.

All of the GID-containing Xaxin deletion mutants described in this example which bound with GSK-3 beta also inhibited GSK-3 beta, indicating that GID binding with GSK-3 beta is required for inhibition. Full length axin is able to bind with GSK-3 beta protein which lacks as many as 62 of its amino-terminal amino acid resides or as many as 132 of its carboxyl-terminal amino acid residues, indicating that axin binds with the catalytic domain of GSK-3 beta.

The GSK-3 Beta Interaction Domain of Axin Binds, but Does Not Inhibit, GSK-3 Beta In Vitro

Full length Xaxin and the deletion mutants comprising the GID of Xaxin exhibit fundamentally different activities in vivo, as described herein. Others have reported that a region of rat axin including the GID and the beta-catenin interaction domain thereof (i.e. amino acid residues 289-506) promoted GSK-3 beta mediated phosphorylation of beta-catenin in vitro (Ikeda et al., 1998, EMBO J. 17:1371-1384). Because the amino acid sequence of this rat axin region is analogous to the amino acid sequence of a corresponding portion of Xaxin, including a portion of Xaxin that inhibits GSK-3 beta in vivo (e.g. Xaxin fragments GID-1 and GID-2), a his-tagged GID (GID-2/his) protein fragment was expressed in and purified from E. coli and used to investigate whether the GID of Xenopus axin inhibits GSK-3 beta activity in vitro.

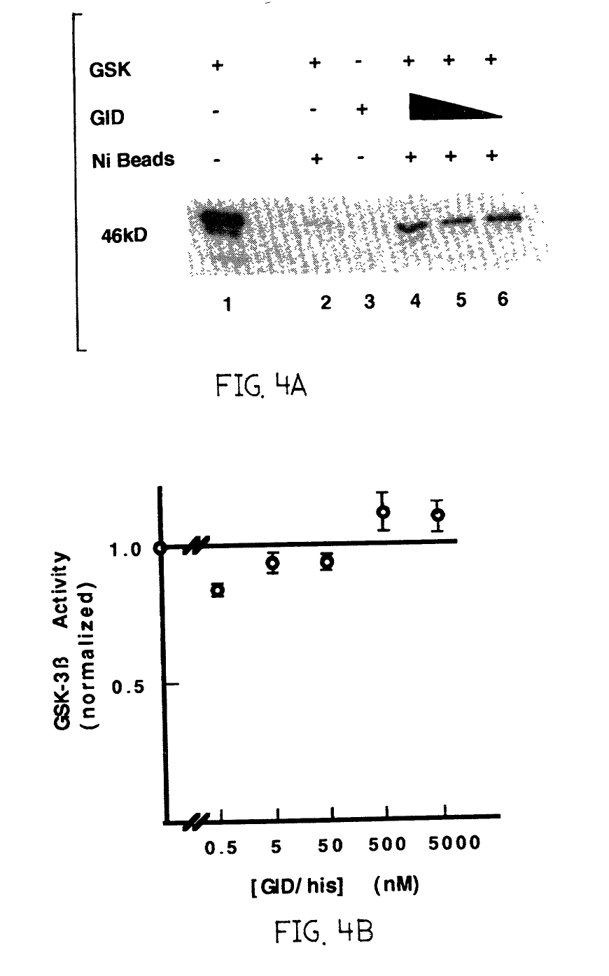

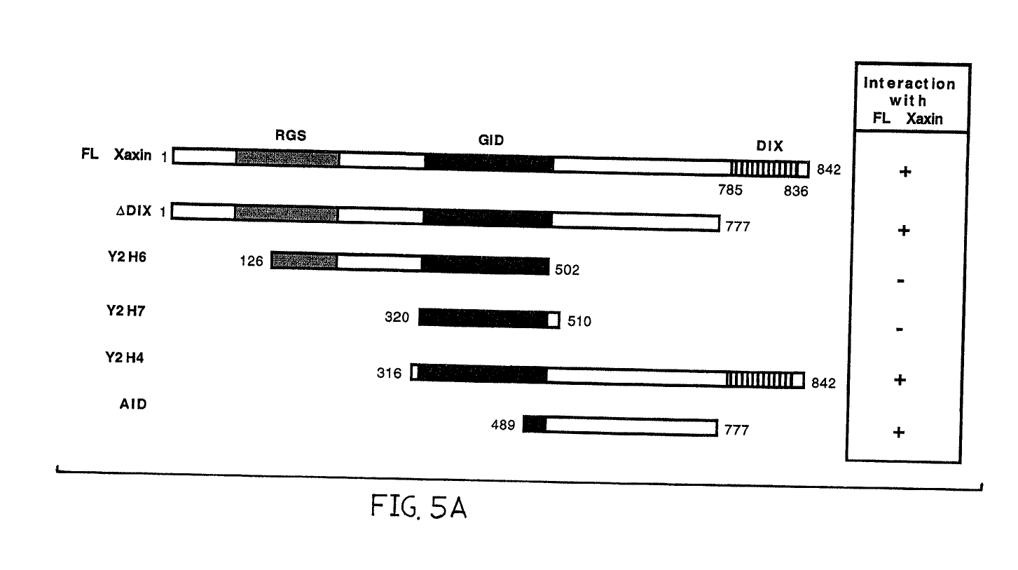

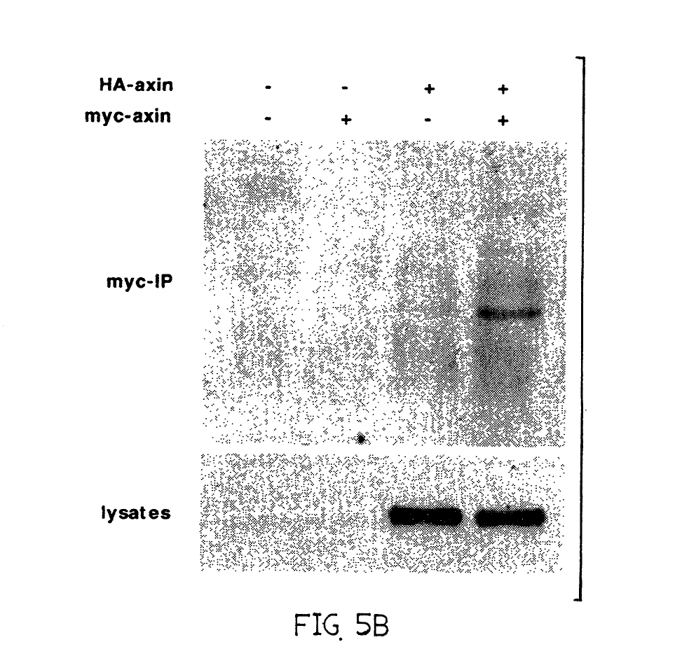

GSK-3 beta (25 nanomolar) was incubated with GID-2/his (up to 200-fold molar excess), and each mixture was assayed to detect either protein kinase activity or, in parallel, protein-protein interaction. Interaction between GSK-3 beta and GID-2/his was detected by purification on nickel agarose followed by immunoblotting with GSK-3 beta-specific antibodies. GSK-3 beta bound specifically to GID-2/his, as indicated in FIG. 4A, lanes 4-6. However, GID-2/his exhibited no significant effect on GSK-3 beta mediated phosphorylation of the GS-2 peptide derived from glycogen synthase, as indicated in FIG. 4B. GID-2/his also did not inhibit phosphorylation of tau by GSK-3 beta, even when GID-2/his was present at a 200-fold molar excess. These results, considered together with the results of Ikeda et al., indicate that the GID binds directly with GSK-3 beta but does not inhibit GSK-3 beta activity in vitro. In contrast, as described herein, the GID robustly inhibits GSK-3 beta activity in oocytes and embryos. These observations indicate that one or more additional factors which are present in vivo are required to inhibit GSK-3 beta bound with the GID of axin.

Identification of an Axin Self-interaction Domain (AID)

Using the yeast two hybrid assay, the ability of axin to bind with itself, with other members of the wnt pathway, or with the G(alpha)q subunit of heterotrimeric G-proteins, was investigated. Full length (FL) Xaxin was cloned into the bait vector as a fusion with the GAL4 DNA binding domain. FL Xaxin, various axin fragments, or other genes were cloned into individual target vectors as fusion proteins with the GAL4 activation domain. These vectors are shown diagrammatically in FIG. 5A.

Saccharomyces cerevisiae cells were transformed with the bait vector and with one of the target vectors. Interaction was assessed using a filter assay to detect beta-galactosidase activity, as described (Fields et al., 1989, Nature 340:245-247; Harper et al., 1993, Cell 75:805-816). Axin did not interact with disheveled, with carboxyl-terminal fragments of Xenopus frizzled 3 and 7, or with G(alpha)q. However, FL Xaxin interacted strongly with GSK-3 beta and with itself. The axin deletion constructs illustrated in FIG. 5A were used to identify the location of the AID of axin. The AID is contained within amino acid residues 489-777 of Xaxin, as evidenced by the fact that this region is sufficient to mediate axin self-interaction. Axin also interacts with itself in Xenopus embryos, as detected by co-immunoprecipitation of myc-tagged and HA-tagged axin, as shown in Figure SB. Thus, axin appears to interact with a number of proteins including itself, APC, GSK-3 beta, and beta-catenin. Others have reported that axin interacts with itself in two-hybrid assays and in co-immunoprecipitations; however that work identified a distinct interaction domain lying within the DIX domain (Hsu et al., 1999, J. Biol. Chem. 274 274:3439-3445).

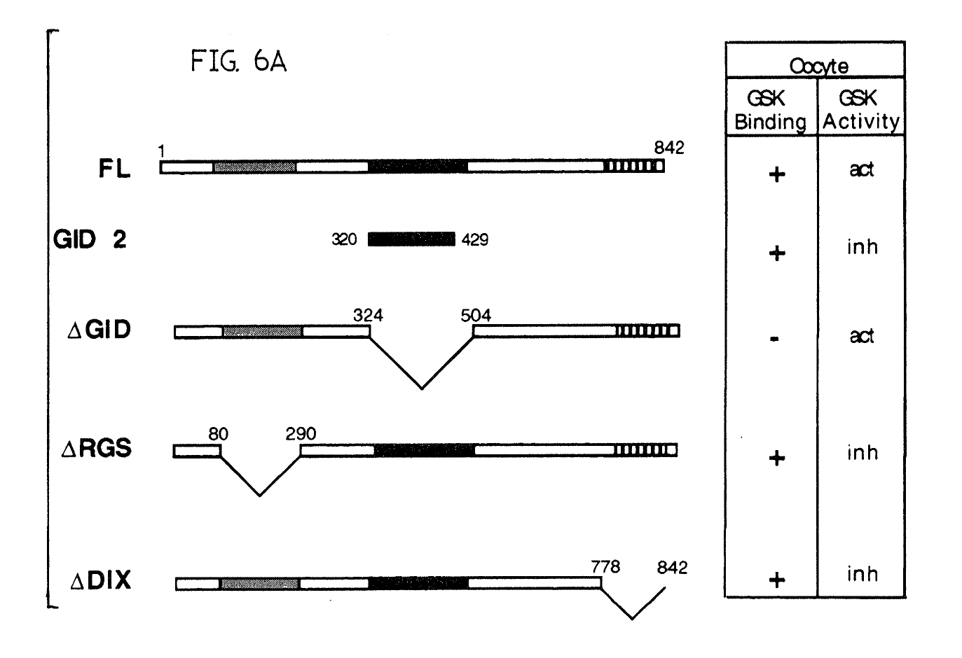

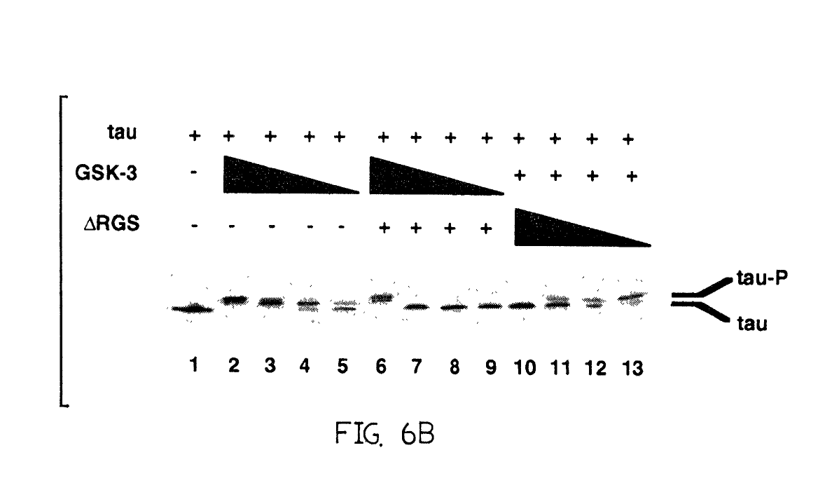

Without being bound by any particular theory of operation, the following explanation for the inhibition of GSK-3 beta described in this example by deletion mutants of axin, but not by full length axin, is presented. It may be that axin complex formation (e.g. association of axin, APC, beta-catenin, and GSK-3 beta) is essential to maintain the activity of GSK-3 beta bound to axin and to ensure normal dorsal-ventral development in the embryo. As described in this example, two axin molecules interact with one another at a region (i.e. the AID) lying between the beta-catenin binding site and the DIX domain. It may be that mutations that disrupt axin complex formation will lead to in vivo inhibition of GSK-3 beta. For example, the deltaRGS mutant, a potent in vivo inhibitor of GSK-3 beta (as indicated in FIG. 6B), does not bind APC. The effect of deltaRGS may thus be functionally equivalent to loss of APC, which results in marked accumulation of beta-catenin protein in colonic epithelia, presumably due to inhibition of GSK-3 beta-mediated phosphorylation of beta-catenin (Rubinfeld et al., 1993, Science 262:1731-1734). Rescue of GSK-3 beta activity and normal ventral axis formation mediated by co-expression of a deltaRGS construct with a deltaGID construct, as described in this example, can thus be explained by assembly of deltaRGS (GSK-3 beta-binding) and deltaGID (APC-binding) constructs by way of the AID. Notably, an amino-terminal fragment of axin containing the RGS domain but lacking the AID does not rescue activity, and axin fragments comprising the GID, but not the RGS or self-interaction domains, are not rescued by deltaGID.

Thus, according to the model presented herein, axin complex formation, involving both homomeric (e.g. axin-axin) and heteromeric (e.g. interactions between axin and one or more of APC, beta-catenin, and GSK-3 beta) interactions, is required to maintain GSK-3 beta activity in vivo.

Axin Complex Formation and Wnt Signaling

Because the Xenopus GID inhibited GSK-3 beta activity, various Xaxin deletion mutants were constructed in order to determine which exhibited analogous inhibitory activity. A Xaxin deletion construct designated deltaGID lacked amino acid residues 324-504, and therefore lacked both the GSK-3 beta and beta-catenin binding sites of full length Xaxin. A Xaxin deletion construct designated deltaRGS lacked the RGS domain of full length Xaxin, which binds APC protein, and is analogous to a mouse deltaRGS mutant described previously (Zeng et al., 1997, Cell 90:181-192). A Xaxin deletion construct designated deltaDIX lacked the 64 carboxyl-terminal amino acid residues of fiull length Xaxin, and therefore lacked the disheveled homology domain.

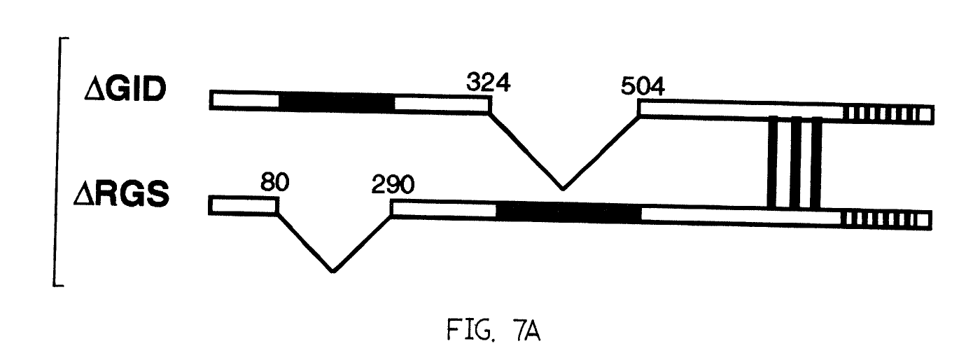

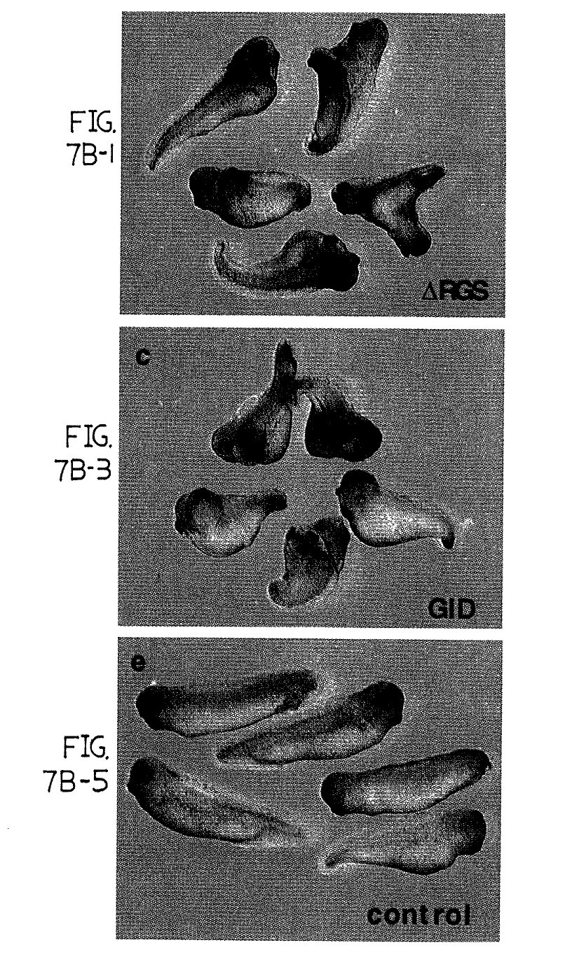

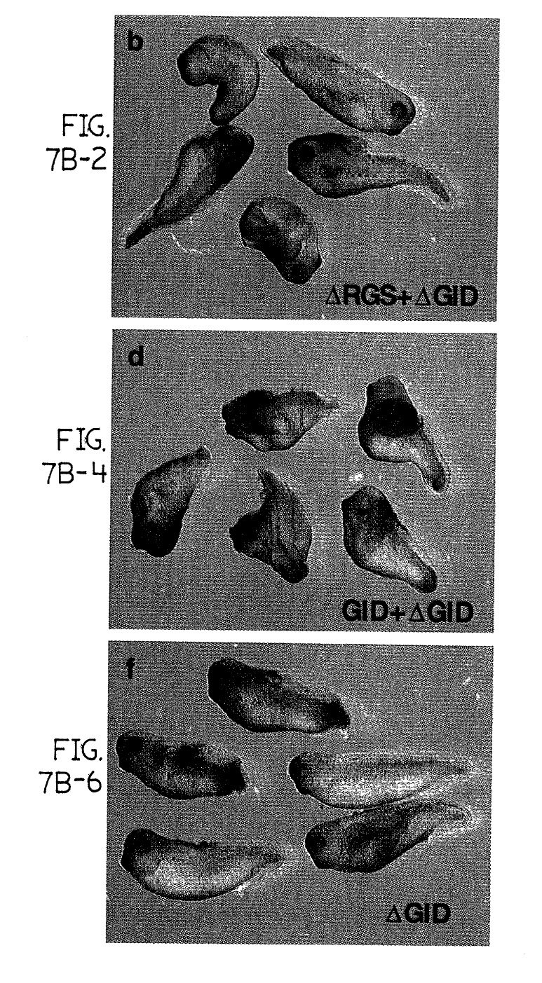

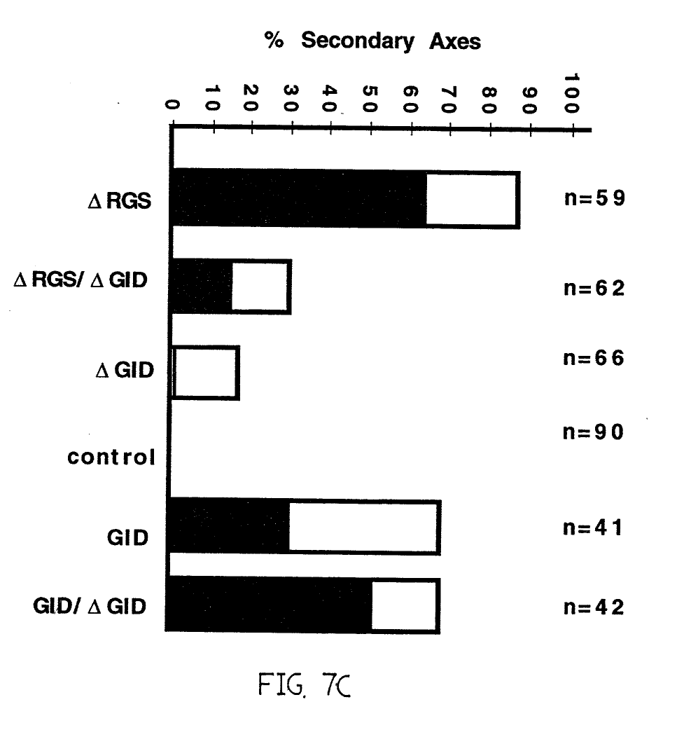

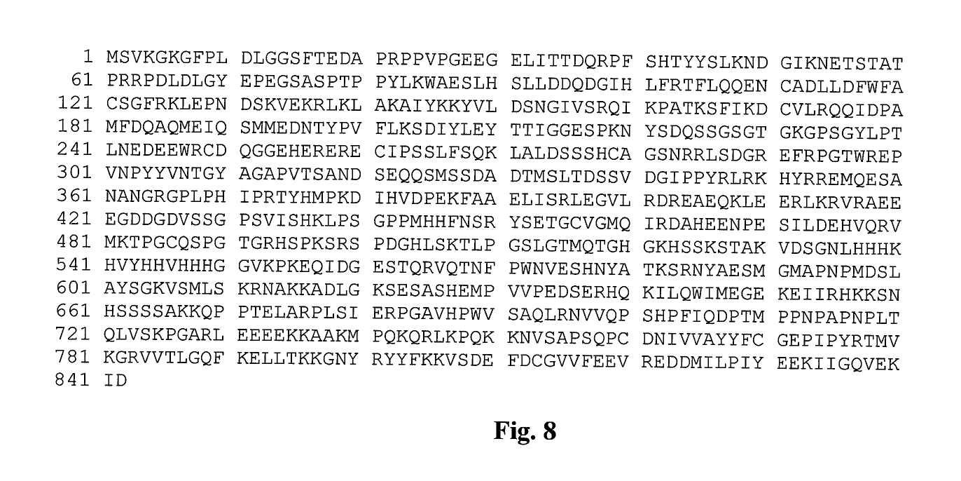

DeltaRGS bound with GSK-3 beta (as indicated in FIG. 6A) and inhibited tau protein phosphorylation mediated by GSK-3 beta in a dose-dependent manner (as indicated in FIG. 6B, lanes 6-13). Inhibition by a fixed concentration of deltaRGS could be overcome by increasing the level of GSK-3 beta (as indicated in FIG. 6B, lanes 6-9). This inhibition was similar to inhibition of GSK-3 beta activity observed using the GID of Xaxin (i.e. as shown in FIGS. 1 and 2) or lithium (Hedgepeth et al., 1997, Develop. Biol. 185:82-91). Inhibition of GSK-3 beta activity by deltaRGS may also explain the dorsalizing activity of the mouse deltaRGS mutant (Zeng et al., 1997, Cell 90:181-192), and the dorsalizing activity of Xenopus deltaRGS (as shown in FIG. 7).

The deltaDIX mutant also bound with GSK-3 beta and partially inhibited GSK-3 beta activity. These observations indicate that the presence of the RGS and DIX domains, or the presence of proteins that bind to these domains, is necessary for GSK-3 beta activity and normal axis formation.

The deltaGID construct did not bind with GSK-3 beta and had no discernible effect on GSK-3 beta activity in the tau-phosphorylation assay, as indicated in FIG. 6A. These observations indicate that the GID is both necessary and sufficient for in vivo binding and inhibition of GSK-3 beta by axin mutants.

As described above, deletion mutants of axin that bind GSK-3 beta inhibit its enzymatic activity in vivo, yet full length axin, which also binds GSK-3 beta, does not inhibit its activity. Without being bound by any particular theory of operation, the inventors recognize that at least two general mechanisms could explain this difference. First, deletion of domains such as the RGS or DIX domains could allow axin mutants to become inhibitory in vivo. Second, the presence of these domains could protect GSK-3 beta from inhibition, for example by recruiting additional proteins, such as APC, into the axin complex.

Occurrence of the AID in Xaxin was used to determine whether a functional axin-GSK-3 beta complex could be reconstituted in vivo from deltaRGS and deltaGID mutant constructs. The inhibitory deltaRGS mutant was co-expressed in Xenopus embryos with the deltaGID mutant which, as described above, does not bind with GSK-3 beta or affect its activity. Although the deltaRGS mutant potently induces dorsalization of embryos (as shown in FIGS. 7B and C; 86% of embryos exhibited dorsalization, n=59), embryos expressing both deltaGID and deltaRGS mutants displayed a marked reduction in the frequency and extent of secondary axes (30% of embryos exhibited dorsalization, n=62). Dorsalization induced by expression of the GID, which lacks the self-interaction domain, was not rescued by deltaGID (as shown in FIGS. 7B and C). Dorsalization induced by expression of the GID was also not rescued by co-expression with an amino-terminal fragment of Xaxin that included the RGS domain. Thus, deltaGID specifically rescues the dominant inhibitory effects of deltaRGS. These observations indicate that self-interaction allows recruitment of one or more cellular factors that prevent inhibition of GSK-3 beta. These experiments indicate the importance of axin complex formation to prevent inhibition of GSK-3 beta and to maintain ventral cell fate.

In order to test the activity of GID fragments in mammalian cells, nucleic acids from which GID fragments could be expressed were transfected into 293T and neuro 2A cells, and the transfected cells were assayed. Stabilization of beta-catenin is now used as a standard assay for activation of wnt signaling. When the GID plasmid was transfected into neuro 2A or 293T cells, beta-catenin levels were markedly increased, as determined by western blotting. This effect was similar to that observed following treatment with lithium.

GID fragments are highly potent activators of the LEF-luciferase reporter construct described by others (Cox et al., 1971, Nature 232: 336-338), but not a control reporter which lacked the LEF binding sites. Single plasmids encoding individual GID fragments were transfected into 293T cells, together with the LEF-luciferase reporter, and luciferase activity was measured after 24 hours. GID fragments induced up to 100- to 250-fold activation of LEF-luciferase (normalized to activity from the mutated LEF-reporter control, which was activated less than 2-fold). Expression of the GID construct also inhibited GSK-3 beta mediated phosphorylation of tau protein in neuro 2A cells, an effect similar to lithium-treatment of cells. This indicates that GID peptide inhibits the enzymatic activity of GSK-3 beta and that its inhibitory action is not limited to activation of the wnt pathway.

An antibody which binds specifically with the GID domain has been generated, using standard antibody generation methods. In one embodiment, the antibody is a high titer polyclonal rabbit antibody that cross-reacts with mouse, rat, and human axin. The antibody reacts in western blots and immunoprecipitations and is can be used in immunohistochemical staining in order to localize axin protein or to inhibit interaction of axin and GSK-3 beta.

Analysis of GID residues required for GSK-3 beta binding and inhibition was performed using a screening method in yeast which is a variation on the reverse two hybrid approach. In this approach, a 300 residue GID-encoding nucleotide sequence (designated GID 1-2) was randomly mutagenized and cells transfected with the nucleotide sequence in an expression vector were screened for mutants that failed to interact with GSK-3 beta. Non-interacting clones were identified by poor growth on histidine (histidine auxotrophy provides a selective pressure for positive interaction in the yeast two hybrid screen, thus non-interactors should grow poorly in the absence of histidine) and by failure to express beta-galactosidase.

Initially, 30 independent clones were identified, but in each case the failure to interact could be attributed to premature stop codons upstream of the known minimal GSK-3 beta interaction sequence. Since these mutants were uninformative, the screen was repeated using temperature sensitive growth in the absence of histidine. It was reasoned that if interaction were temperature sensitive, then a functional GID sequence must be present at the permissive temperature, and it would be more likely for this to arise due to a point mutation.

1000 clones were isolated, replica plated onto histidine-free plates and cultured at 16°, 23°, 30°, and 37° C. From this analysis, 16 non-interacting clones were identified and 14 of them were sequenced. Three clones contained point mutations within the 25 amino acid residue GID sequence, and the other 11 exhibited premature termination. Of the three point mutations, two were independent isolates of a similar mutation that converts an absolutely conserved arginine residue (arg-403) to a proline residue. The other mutant replaced phenylalanine-388 with a tyrosine residue (phe-388 is also completely conserved among vertebrate axins). The phe-388 to tyr (GID5-6FY) was tested in the LEF-luciferase assay, and its activity in this assay (at 37°) was reduced approximately 10-fold compared to wild-type.

The experiments presented in this example demonstrate that deletion mutants of Xaxin that include the GID antagonize the activity of GSK-3 beta in vivo. Such mutant polypeptides can be used to activate Wnt signaling in an organism by providing one of the polypeptides to the organism.

The experiments presented in this example also demonstrate that axin is capable of interacting with itself and that a multimeric axin complex is required to maintain GSK-3 beta activity in vivo and thus to antagonize Wnt signaling. These data indicate that axin, in addition to facilitating beta-catenin phosphorylation mediated by GSK-3 beta, can also mediate inhibition of GSK-3 beta in response to extracellular signals such as Wnts.

### EXAMPLE 2

Generation of Transgenic Mice Encoding Axin GID in an Inducible Construct

Because GSK-3 beta and the wnt signaling pathway have central roles during early development of a number of tissues, including the central nervous system, it is critical to restrict expression of the transgenes both spatially and temporally in transgenic mice which are intended to express GSK-3 beta or other wnt signaling proteins aberrantly. The calcium-calmodulin dependent protein kinase II alpha (CaMKIIalpha) promoter drives expression of transgenes only in the forebrain (neocortex, hippocampus, amygdala, and basal ganglia) and only post-natally through adulthood. This promoter has proven to be a powerful tool for studying the role of important signaling molecules, such as CaMKIIalpha itself, as well as the NMDA receptor, in memory and learning in adult mice (see, e.g., Greenberg et al., 1992, J. Biol. Chem 267:564-569). It has also been successfully used to derive transgenic mouse lines that express dominant negative GSK-3 beta in the cortex. Overt developmental abnormalities were not observed in those mice. Transgenes can be constructed with a hybrid intron/exon in order to improve expression, as described (Bramblett et al., 1993, Neuron 10:1089-1099). In one embodiment, the vector used to transform mice contains about 8.0 kilobases of DNA upstream of the CaMKIIalpha transcription start site as well as 85 base-pairs of the 5′ non-coding exon, a 5′ intron, a second exon with a cloning site for cDNA insertion, and the SV40 3′ intron and polyadenylation signal.

Specific transgenes that can be used to generate transgenic mice include myc tagged GID5-6 (i.e., including a detectable tag which does affect GID activity) and inactive GID forms, such as GID5-6FY. These sequences can be derived from Xenopus axin, since the amino acid sequence of the Xenopus GSK interaction domain is highly similar to mammalian axin and the Xenopus derived peptide is well characterized as a potent inhibitor of GSK-3 beta in mouse and human cell lines.

DNA injections and establishment of founders is performed as follows. About 150 fertilized eggs are injected initially, and 2-cell embryos are implanted in pseudopregnant foster females. Typically, about 40 viable offspring are obtained and about 15% of these are transgenic. For routine transgenic experiments, approximately three founders can be expected to exhibit adequate expression and to be able to breed. Founders are back-crossed into the C57BL/6 strain to establish stable lines in a strain that is well characterized in behavioral assays.

Progeny are assessed initially either by Southern blotting or by PCR analysis of genomic DNA isolated from tail biopsies in order to establish their genotype. Expression of the transgenes is measured in progeny by reverse transcriptase-PCR and Northern analysis of brain tissue. Primer sequences and RNA probes are based on sequences unique to the transgene, including the hybrid exons and regions of the transgene DNA sequence that are sufficiently distinct from the mouse sequence to avoid cross-hybridization. Subsequently, tissue specific gene expression is determined by in situ hybridization in brain sections. The level of protein expression is determined by Western blot of whole brain extract and by immunohistochemical staining of sections with epitope specific antibodies.

In vivo activity of GSK-3 can be determined by assessing phosphorylation of tau protein using phosphorylation-specific antibodies, such as the antibody designated PHF-1 in the art, in immunohistochemistry. In addition, gross neuroanatomy and histology of Nissl stained brain sections derived from transgenic animals from each established line can be examined at autopsy to rule out significant anatomic defects that reflect perturbation of early development.

Once lines are established that express these transgenes, mice are back-crossed into C57B6 and then subjected, together with non-transgenic littermates, to a battery of behavioral tests that are known in the art. Such tests include, for example, the forced swim test, the open field test, and the acoustic startle with pre-pulse inhibition test. Multiple mice (e.g., 10 mice per line) should be tested per experimental group, in order to enhance reproducibility and confidence.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

What is claimed is:

1. A composition that inhibits glycogen synthase kinase 3 beta activity, the composition comprising a polypeptide of not more than about 60 amino acid residues, the polypeptide having an amino acid sequence which comprises the sequence
Val-Xaa₅-Pro-Xaa₇-Xaa₈-Phe-Ala-Xaa₁₁-Glu-Leu-Ile-Xaa₁₅-Arg-Leu-Glu-Xaa₁₉-Xaa₂₀-Xaa₂₁-Xaa₂₂-Xaa₂₃-Xaa₂₄-Glu,
wherein
each of Xaa₇, Xaa₈, Xaa₁₁, and Xaa₁₉ is independently any amino acid residue,
Xaa₅ is a negatively-charged amino acid residue,
Xaa₁₅ is a polar amino acid residue,
Xaa₂₀ is a non-polar aliphatic amino acid residue, and
at least two of Xaa₂₁, Xaa₂₂, Xaa₂₃, Xaa₂₄ are polar amino acid residues, the balance of Xaa₂₁, Xaa₂₂, Xaa₂₃, Xaa₂₄ being any amino acid residue.

2. The composition of claim 1, wherein at least two of Xaa₂₁, Xaa₂₂, Xaa₂₃, Xaa₂₄ are charged amino acid residues.

3. The composition of claim 2, wherein Xaa₇ is a polar amino acid residue, Xaa₈ is Lys, Xaa₂₀ is Val, Xaa₂₁ is any amino acid residue, Xaa₂₂ is a positively-charged amino acid residue, Xaa₂₃ is a polar amino acid residue, and Xaa₂₄ is Arg.

4. The composition of claim 3, wherein the amino acid sequence comprises the sequence
Xaa₁-Xaa₂-Xaa₃-Val-Xaa₅-Pro-Xaa₇-Xaa₈-Phe-Ala-Xaa₁₁-Glu-Leu-Ile-Xaa₁₅-Arg-Leu-Glu-Xaa₁₉-Xaa₂₀-Xaa₂₁-Xaa₂₂-Xaa₂₃-Xaa₂₄-Glu,
wherein
each of Xaa₁, Xaa₂, and Xaa₃ is independently any amino acid residue.

5. The composition of claim 4, wherein Xaa₁ is a negatively-charged amino acid residue, Xaa₂ is a non-polar amino acid residue, and Xaa₃ is a positively-charged amino acid residue.

6. The composition of claim 4, wherein
Xaa₁ is selected from the group consisting of Asp, Glu, and Met;
Xaa₂ is selected from the group consisting of Ile, Val, and Thr;
Xaa₃ is selected from the group consisting of His, Arg, and Pro;
Xaa₅ is selected from the group consisting of Asp and Glu;
Xaa₇ is selected from the group consisting of Glu, Gln, and Ala;
Xaa₈ is selected from the group consisting of Lys, Thr, and Ala;
Xaa₁₁ is selected from the group consisting of Ala and Glu;
Xaa₁₅ is selected from the group consisting of Ser, Asn, and His;
Xaa₁₉ is selected from the group consisting of Gly, Glu, Ala, and Lys;
Xaa₂₀ is selected from the group consisting of Val and Leu;
Xaa21 is selected from the group consisting of Leu, Gln, and Lys;
Xaa₂₂ is selected from the group consisting of Arg, Lys, and Leu;
Xaa₂₃ is selected from the group consisting of Asp, Glu, and Thr; and
Xaa₂₄ is selected from the group consisting of Arg and Leu.

7. The composition of claim 6, wherein the sequence is selected from the group consisting of SEQ ID NOs: 1-7.

8. The composition of claim 6, wherein
Xaa₁ is selected from the group consisting of Asp and Glun;
Xaa₂ is selected from the group consisting of Ile and Val;
Xaa₃ is selected from the group consisting of His and Arg;
Xaa₇ is selected from the group consisting of Glu and Gln;
Xaa₈ is Lys;
Xaa₁₉ is selected from the group consisting of Gly, Glu, and Ala;
Xaa₂₀ is Val;
Xaa₂₁ is selected from the group consisting of Leu and Gln;
Xaa₂₂ is selected from the group consisting of Arg and Lys; and
Xaa₂₄ is Arg.

9. The composition of claim 8, wherein the sequence is selected from the group consisting of SEQ ID NOs: 1-4.

10. The composition of claim 1, wherein the polypeptide comprises not more than about 30 amino acid residues.

11. The composition of claim 1, further comprising a pharmaceutically acceptable carrier.

12. A kit for inhibiting glycogen synthase kinase 3 beta activity, the kit comprising the composition of claim 1 and an instructional material selected from the group consisting of an instructional material that describes administration of the composition to an animal in order to inhibit glycogen synthase kinase 3 beta activity, an instructional material that describes administration of the composition to an animal in order to activate wnt signaling, an instructional material that describes administration of the composition to an animal in order to alleviate a disorder known to be alleviated by administration of lithium, and an instructional material that describes administration of the composition to a mammal in order to inhibit spermatozoal motility.

13. A method of inhibiting glycogen synthase kinase 3 beta activity in an animal, the method comprising administering the composition of claim 1 to the animal.

14. A method of activating wnt signaling in an animal, the method comprising administering the composition of claim 1 to the animal.

15. A method of alleviating, in an animal, a disorder known to be alleviated by administration of lithium, the method comprising administering the composition of claim 1 to the animal.

16. The method of claim 15, wherein the disorder is selected from the group consisting of bipolar disorder, mania, depression, Alzheimer's disease, diabetes, and leukopenia.

17. A method of inhibiting motility of mammalian spermatozoa, the method comprising contacting the composition of claim 1 and the spermatozoa, whereby spermatozoal motility is inhibited.

18. A method of inhibiting phosphorylation of a protein in a cell, wherein the protein is selected from the group consisting of beta-catenin, glycogen synthase, phosphatase inhibitor I-2, the type-II subunit of cAMP-dependent protein kinase, the G-subunit of phosphatase-1, ATP-citrate lyase, acetyl coenzyme A carboxylase, myelin basic protein, a microtubule-associated protein, a neurofilament protein, an N-CAM cell adhesion molecule, nerve growth factor receptor, c-Jun transcription factor, JunD transcription factor, c-Myb transcription factor, c-Myc transcription factor, L-myc transcription factor, adenomatous polyposis coli tumor suppressor protein, and tau protein, the method comprising providing the composition of claim 1 to the cell.

19. A composition that inhibits glycogen synthase kinase 3 beta activity in vivo, the composition comprising a polypeptide having the amino acid sequence of at least a portion of the region between the GID domain and the DIX domain of an axin.

20. The composition of claim 19, wherein the polypeptide has the amino acid sequence of at least a portion of residues 489-777 of SEQ ID NO: 8.

21. The composition of claim 20, wherein the polypeptide has the amino acid sequence of residues 489-777 of SEQ ID NO: 8.

22. A method of alleviating, in an animal, a disorder known to be alleviated by administration of lithium, the method comprising administering the composition of claim 19 to the animal.

23. A method of identifying an inhibitor of glycogen synthase kinase 3 beta activity, the method comprising assessing glycogen synthase kinase 3 beta activity in an assay system in the presence and absence a polypeptide having an amino acid sequence that consists of less than all of the sequence between the GID domain and the DIX domain of an axin, whereby the polypeptide is an inhibitor of glycogen synthase kinase 3 beta activity if the activity in the assay system is greater in the absence of the polypeptide than in the presence of the polypeptide.

24. The method of claim 23, wherein the polypeptide has an amino acid sequence that consists of less than all of residues 489-777 of SEQ ID NO: 8.

25. An antibody that binds specifically with a polypeptide having an amino acid sequence which comprises the sequence
Val-Xaa₅-Pro-Xaa₇-Xaag-Phe-Ala-Xaa₁₁-Glu-Leu-Ile-Xaa₁₅-Arg-Leu-Glu-Xaa₁₉-Xaa₂₀-Xaa₂₁-Xaa₂₂-Xaa₂₃-Xaa₂₄-Glu,
wherein
each of Xaa₇, Xaa₈, Xaa₁₁, and Xaa₁₉ is independently any amino acid residue,
Xaa₅ is a negatively-charged amino acid residue,
Xaa₁₅ is a polar amino acid residue,
Xaa₂₀ is a non-polar aliphatic amino acid residue, and
at least two of Xaa₂₁, Xaa₂₂, Xaa₂₃, Xaa₂₄ are polar amino acid residues, the balance of Xaa₂₁, Xaa₂₂, Xaa₂₃, Xaa₂₄ being any amino acid residue.

26. The antibody of claim 25, wherein the sequence is selected from the group consisting of SEQ ID NOs: 1-7.

27. A method of alleviating, in an animal, a disorder known to be alleviated by administration of lithium, the method comprising administering to the animal the antibody of claim 25.

28. A transgenic animal comprising an expressible transgene, wherein the transgene encodes a polypeptide of not more than about 60 amino acid residues, the polypeptide having an amino acid sequence which comprises the sequence
Val-Xaa₅-Pro-Xaa₇-Xaa₈-Phe-Ala-Xaa₁₁-Glu-Leu-Ile-Xaa₁₅-Arg-Leu-Glu-Xaa₁₉-Xaa₂₀-Xaa₂₁-Xaa₂₂-Xaa₂₃-Xaa₂₄-Glu,
wherein
each of Xaa₇, Xaa₈, Xaa₁₁ and Xaa₁₉ is independently any amino acid residue,
Xaa₅ is a negatively-charged amino acid residue,
Xaa₁₅ is a polar amino acid residue,
Xaa20 is a non-polar aliphatic amino acid residue, and
at least two of Xaa₂₁, Xaa₂₂, Xaa₂₃, Xaa₂₄ are polar amino acid residues, the balance of Xaa₂₁, Xaa₂₂, Xaa₂₃, Xaa₂₄ being any amino acid residue.

29. The transgenic animal of claim 28, wherein the sequence is selected from the group consisting of SEQ ID NOs: 1-7.

30. The transgenic animal of claim 28, wherein the polypeptide binds with glycogen synthase 3 beta.

31. The transgenic animal of claim 28, wherein the polypeptide inhibits glycogen synthase 3 beta activity.

32. The transgenic animal of claim 28, wherein the animal is a mouse.

33. The transgenic animal of claim 28, wherein the transgene comprises an inducible promoter operably linked with the portion of the transgene encoding the polypeptide.
